SEQUENCE LISTING (29 sequences)

SEQ ID NO: 1 - DNA, 18 bp, Artificial Sequence
  synthetic oligonucleotide
ggaaatgctgttccaagg

SEQ ID NO: 2 - DNA, 20 bp, Artificial Sequence
  synthetic oligonucleotide
gcacagttgccatcattgtc

SEQ ID NO: 3 - DNA, 30 bp, Artificial Sequence
  synthetic oligonucleotide
tggaaggcatgtcagtgccaaaagatgagg

SEQ ID NO: 4 - DNA, 32 bp, Artificial Sequence
  synthetic oligonucleotide
gtagatcctgcgtgttgtgtagtccagaacaa

SEQ ID NO: 5 - DNA, 32 bp, Artificial Sequence
  synthetic oligonucleotide
gtagatcctgcgtgttgtgtagtccagaacag

SEQ ID NO: 6 - DNA, 22 bp, Artificial Sequence
  synthetic oligonucleotide
tcttggcgggacgaacctccga

SEQ ID NO: 7 - DNA, 22 bp, Artificial Sequence
  synthetic oligonucleotide
acacacatctgtaggattaaat

SEQ ID NO: 8 - DNA, 20 bp, Artificial Sequence
  synthetic oligonucleotide
ttgcaatagtcagcatgaag

SEQ ID NO: 9 - DNA, 22 bp, Artificial Sequence
  synthetic oligonucleotide
tcttggcgggacaaacctgagg

SEQ ID NO: 10 - DNA, 22 bp, Artificial Sequence
  synthetic oligonucleotide
acacacatctgcaggattaaac

SEQ ID NO: 11 - DNA, 20 bp, Artificial Sequence
  synthetic oligonucleotide
tggcaatagttagcatgaag

SEQ ID NO: 12 - DNA, 5313 bp, homo sapiens
agtctggaattcagatgcctattggtgactgctccgtggcagctaaaccaagaaaacagctgctctgctcattatttcaaaccacactagggtacagagctcgtgcttcgggatggaaacctatggttatctgcagagggagtcatgctttcaaggacctcatgaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgcctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtcccagctactcgggaggctgaggcaggagaatggcatgaaccagggaggcggagctggcagtgaactgagaccgcaccactgcactccagcctgggtgacagagcaagactccatctcaaaaataataataataaataataataataataataataataataataatccttccagctgggcgcagtggctcacgcctgtaatcccaacattttgggaggccgagatgggcggatcacctgaggtcaggagttcaagaccagcctggccaacatggtgaaaccccatctctactaaaatacaaaaattagccgggcgtggtggcatgtgcctgtagtcccagctactcgggaggctgaggcaggagaattgcttgaacccgggaggcggaggttgcagtgagccgagatcatgccactgcactccaccctgggtgacagagcgagactccgtctacacacacacacacacacacacacacatccttcctcctctaaccccaaactaagatcacagaaggtgatccagtcagagaacagagggaaatcttaccaggaagggcttaagtacacttttttttaaaacagctttattgtttttaaagcctacaatttgataagccttgacatatgtatacctgtgaaagcatcaccacaatcaagacactggacatatctatcactcccccatctcagatatcccccctaatcctggataccatttgttgaaagatgttattactctagctgaacttacaagagactttagaccagggatctaaattacagtggccttagtgaccttgtccttatcttcttaggacagctgagaagccactgggacttagagcctttaaaaggagattaactgtcccaaaaggatctttgctactgaccagcagacacttcttccttcagtagcctttcatactgtgttgagtaacaccctagggtgtccattaaagttttgagttttacctagagcccagagccatgaatcaggattctgtctacatgattcgtgttttcattggtgtcaaaatacaaaagccaaagttctggctatgaattgttaacttggaagaaatactaactgccaccacttattaagtgcctactgtgtgccaggctctgaactaggtgcttcatatacattatcctaaattatctcaacatatgaggtaggtgttttaatttttattttatagaacttggtgtgtttgactgttaagctatggggctagagagagggtttgatcccaggtccctctgtgcttttgctgctgagccacacaacctctcatttcaaaaacactttcaaaatgctaacatattctaattcactctaggccaccaaaaactttaatactaatatctgatttgtaaatgacttaatgtatccttgaccctatcagctgaatttaatgaaatattcctctctgctgtgaaattttaccagtatagtatttggtctagtgacaggtgttctatttttttgagacgggtctcactctgtcacccaggctggagtgcagtggctcaatgcaacctctgccccccaagttcaagtgattctcctgcctcagcctcttgagtagctgggattacaggcacatgcaccacgcccggctgatttttttttgtatttttagtagacagggtttcaccatgttggccaggctggtcttgaactcctgacctcaggtgatccgcccgcctctgcctcccaaagtgctgggattacaggtgtaagccatcacacctggccctagtgacaggtttttatgggtacttttagatgatctaagaaatcatgtgcatatatctttcagatttctattttgggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccagggcctggttattgttcagacattccatggtgattcctgggaaggaagcttggctgctcagtttctgagtctggggtgagataatgttctgggaagggacatctgttctttggtgtaatctctcatggtgaaatctgctctgtacatcagacaattgcattgctaccaagtttcataccaaatatttgaaaggatggtattgaatctaaaaccaaatattagtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaaaaaaaaaa

SEQ ID NO: 13 - DNA, 5107 bp, homo sapiens
gtgggcgtggctgggcgagcgaggagtggggacaaggtcgagcgacgagtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgcctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtcccagctactcgggaggctgaggcaggagaatggcatgaaccagggaggcggagctggcagtgaactgagaccgcaccactgcactccagcctgggtgacagagcaagactccatctcaaaaataataataataaataataataataataataataataataataatccttccagctgggcgcagtggctcacgcctgtaatcccaacattttgggaggccgagatgggcggatcacctgaggtcaggagttcaagaccagcctggccaacatggtgaaaccccatctctactaaaatacaaaaattagccgggcgtggtggcatgtgcctgtagtcccagctactcgggaggctgaggcaggagaattgcttgaacccgggaggcggaggttgcagtgagccgagatcatgccactgcactccaccctgggtgacagagcgagactccgtctacacacacacacacacacacacacacatccttcctcctctaaccccaaactaagatcacagaaggtgatccagtcagagaacagagggaaatcttaccaggaagggcttaagtacacttttttttaaaacagctttattgtttttaaagcctacaatttgataagccttgacatatgtatacctgtgaaagcatcaccacaatcaagacactggacatatctatcactcccccatctcagatatcccccctaatcctggataccatttgttgaaagatgttattactctagctgaacttacaagagactttagaccagggatctaaattacagtggccttagtgaccttgtccttatcttcttaggacagctgagaagccactgggacttagagcctttaaaaggagattaactgtcccaaaaggatctttgctactgaccagcagacacttcttccttcagtagcctttcatactgtgttgagtaacaccctagggtgtccattaaagttttgagttttacctagagcccagagccatgaatcaggattctgtctacatgattcgtgttttcattggtgtcaaaatacaaaagccaaagttctggctatgaattgttaacttggaagaaatactaactgccaccacttattaagtgcctactgtgtgccaggctctgaactaggtgcttcatatacattatcctaaattatctcaacatatgaggtaggtgttttaatttttattttatagaacttggtgtgtttgactgttaagctatggggctagagagagggtttgatcccaggtccctctgtgcttttgctgctgagccacacaacctctcatttcaaaaacactttcaaaatgctaacatattctaattcactctaggccaccaaaaactttaatactaatatctgatttgtaaatgacttaatgtatccttgaccctatcagctgaatttaatgaaatattcctctctgctgtgaaattttaccagtatagtatttggtctagtgacaggtgttctatttttttgagacgggtctcactctgtcacccaggctggagtgcagtggctcaatgcaacctctgccccccaagttcaagtgattctcctgcctcagcctcttgagtagctgggattacaggcacatgcaccacgcccggctgatttttttttgtatttttagtagacagggtttcaccatgttggccaggctggtcttgaactcctgacctcaggtgatccgcccgcctctgcctcccaaagtgctgggattacaggtgtaagccatcacacctggccctagtgacaggtttttatgggtacttttagatgatctaagaaatcatgtgcatatatctttcagatttctattttgggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccagggcctggttattgttcagacattccatggtgattcctgggaaggaagcttggctgctcagtttctgagtctggggtgagataatgttctgggaagggacatctgttctttggtgtaatctctcatggtgaaatctgctctgtacatcagacaattgcattgctaccaagtttcataccaaatatttgaaaggatggtattgaatctaaaaccaaatattagtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaaaaaaaaaa

SEQ ID NO: 14 - DNA, 4970 bp, homo sapiens
gtgggcgtggctgggcgagcgaggagtggggacaaggtcgagcgacgagtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctgaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgcctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtcccagctactcgggaggctgaggcaggagaatggcatgaaccagggaggcggagctggcagtgaactgagaccgcaccactgcactccagcctgggtgacagagcaagactccatctcaaaaataataataataaataataataataataataataataataataatccttccagctgggcgcagtggctcacgcctgtaatcccaacattttgggaggccgagatgggcggatcacctgaggtcaggagttcaagaccagcctggccaacatggtgaaaccccatctctactaaaatacaaaaattagccgggcgtggtggcatgtgcctgtagtcccagctactcgggaggctgaggcaggagaattgcttgaacccgggaggcggaggttgcagtgagccgagatcatgccactgcactccaccctgggtgacagagcgagactccgtctacacacacacacacacacacacacacatccttcctcctctaaccccaaactaagatcacagaaggtgatccagtcagagaacagagggaaatcttaccaggaagggcttaagtacacttttttttaaaacagctttattgtttttaaagcctacaatttgataagccttgacatatgtatacctgtgaaagcatcaccacaatcaagacactggacatatctatcactcccccatctcagatatcccccctaatcctggataccatttgttgaaagatgttattactctagctgaacttacaagagactttagaccagggatctaaattacagtggccttagtgaccttgtccttatcttcttaggacagctgagaagccactgggacttagagcctttaaaaggagattaactgtcccaaaaggatctttgctactgaccagcagacacttcttccttcagtagcctttcatactgtgttgagtaacaccctagggtgtccattaaagttttgagttttacctagagcccagagccatgaatcaggattctgtctacatgattcgtgttttcattggtgtcaaaatacaaaagccaaagttctggctatgaattgttaacttggaagaaatactaactgccaccacttattaagtgcctactgtgtgccaggctctgaactaggtgcttcatatacattatcctaaattatctcaacatatgaggtaggtgttttaatttttattttatagaacttggtgtgtttgactgttaagctatggggctagagagagggtttgatcccaggtccctctgtgcttttgctgctgagccacacaacctctcatttcaaaaacactttcaaaatgctaacatattctaattcactctaggccaccaaaaactttaatactaatatctgatttgtaaatgacttaatgtatccttgaccctatcagctgaatttaatgaaatattcctctctgctgtgaaattttaccagtatagtatttggtctagtgacaggtgttctatttttttgagacgggtctcactctgtcacccaggctggagtgcagtggctcaatgcaacctctgccccccaagttcaagtgattctcctgcctcagcctcttgagtagctgggattacaggcacatgcaccacgcccggctgatttttttttgtatttttagtagacagggtttcaccatgttggccaggctggtcttgaactcctgacctcaggtgatccgcccgcctctgcctcccaaagtgctgggattacaggtgtaagccatcacacctggccctagtgacaggtttttatgggtacttttagatgatctaagaaatcatgtgcatatatctttcagatttctattttgggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccagggcctggttattgttcagacattccatggtgattcctgggaaggaagcttggctgctcagtttctgagtctggggtgagataatgttctgggaagggacatctgttctttggtgtaatctctcatggtgaaatctgctctgtacatcagacaattgcattgctaccaagtttcataccaaatatttgaaaggatggtattgaatctaaaaccaaatattagtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaaaaaaaaaa

SEQ ID NO: 15 - DNA, 5107 bp, homo sapiens
agtctggaattcagatgcctattggtgactgctccgtggcagctaaaccaagaaaacagctgctctgctcattatttcaaaccacactagggtacagagctcgtgcttcgggatggaaacctatggttatctgcagagggagtcatgctttcaaggacctcataaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgcctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtcccagctactcgggaggctgaggcaggagaatggcatgaaccagggaggcggagctggcagtgaactgagaccgcaccactgcactccagcctgggtgacagagcaagactccatctcaaaaataataataataaataataataataataataataataataataatccttccagctgggcgcagtggctcacgcctgtaatcccaacattttgggaggccgagatgggcggatcacctgaggtcaggagttcaagaccagcctggccaacatggtgaaaccccatctctactaaaatacaaaaattagccgggcgtggtggcatgtgcctgtagtcccagctactcgggaggctgaggcaggagaattgcttgaacccgggaggcggaggttgcagtgagccgagatcatgccactgcactccaccctgggtgacagagcgagactccgtctacacacacacacacacacacacacacatccttcctcctctaaccccaaactaagatcacagaaggtgatccagtcagagaacagagggaaatcttaccaggaagggcttaagtacacttttttttaaaacagctttattgtttttaaagcctacaatttgataagccttgacatatgtatacctgtgaaagcatcaccacaatcaagacactggacatatctatcactcccccatctcagatatcccccctaatcctggataccatttgttgaaagatgttattactctagctgaacttacaagagactttagaccagggatctaaattacagtggccttagtgaccttgtccttatcttcttaggacagctgagaagccactgggacttagagcctttaaaaggagattaactgtcccaaaaggatctttgctactgaccagcagacacttcttccttcagtagcctttcatactgtgttgagtaacaccctagggtgtccattaaagttttgagttttacctagagcccagagccatgaatcaggattctgtctacatgattcgtgttttcattggtgtcaaaatacaaaagccaaagttctggctatgaattgttaacttggaagaaatactaactgccaccacttattaagtgcctactgtgtgccaggctctgaactaggtgcttcatatacattatcctaaattatctcaacatatgaggtaggtgttttaatttttattttatagaacttggtgtgtttgactgttaagctatggggctagagagagggtttgatcccaggtccctctgtgcttttgctgctgagccacacaacctctcatttcaaaaacactttcaaaatgctaacatattctaattcactctaggccaccaaaaactttaatactaatatctgatttgtaaatgacttaatgtatccttgaccctatcagctgaatttaatgaaatattcctctctgctgtgaaattttaccagtatagtatttggtctagtgacaggtgttctatttttttgagacgggtctcactctgtcacccaggctggagtgcagtggctcaatgcaacctctgccccccaagttcaagtgattctcctgcctcagcctcttgagtagctgggattacaggcacatgcaccacgcccggctgatttttttttgtatttttagtagacagggtttcaccatgttggccaggctggtcttgaactcctgacctcaggtgatccgcccgcctctgcctcccaaagtgctgggattacaggtgtaagccatcacacctggccctagtgacaggtttttatgggtacttttagatgatctaagaaatcatgtgcatatatctttcagatttctattttgggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccagggcctggttattgttcagacattccatggtgattcctgggaaggaagcttggctgctcagtttctgagtctggggtgagataatgttctgggaagggacatctgttctttggtgtaatctctcatggtgaaatctgctctgtacatcagacaattgcattgctaccaagtttcataccaaatatttgaaaggatggtattgaatctaaaaccaaatattagtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaaaaaaaaaa

SEQ ID NO: 16 - DNA, 5329 bp, homo sapiens
gtgggcgtggctgggcgagcgaggagtggggacaaggtcgagcgacgagtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgcctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtcccagctactcgggaggctgaggcaggagaatggcatgaaccagggaggcggagctggcagtgaactgagaccgcaccactgcactccagcctgggtgacagagcaagactccatctcaaaaataataataataaataataataataataataataataataataatccttccagctgggcgcagtggctcacgcctgtaatcccaacattttgggaggccgagatgggcggatcacctgaggtcaggagttcaagaccagcctggccaacatggtgaaaccccatctctactaaaatacaaaaattagccgggcgtggtggcatgtgcctgtagtcccagctactcgggaggctgaggcaggagaattgcttgaacccgggaggcggaggttgcagtgagccgagatcatgccactgcactccaccctgggtgacagagcgagactccgtctacacacacacacacacacacacacacatccttcctcctctaaccccaaactaagatcacagaaggtgatccagtcagagaacagagggaaatcttaccaggaagggcttaagtacacttttttttaaaacagctttattgtttttaaagcctacaatttgataagccttgacatatgtatacctgtgaaagcatcaccacaatcaagacactggacatatctatcactcccccatctcagatatcccccctaatcctggataccatttgttgaaagatgttattactctagctgaacttacaagagactttagaccagggatctaaattacagtggccttagtgaccttgtccttatcttcttaggacagctgagaagccactgggacttagagcctttaaaaggagattaactgtcccaaaaggatctttgctactgaccagcagacacttcttccttcagtagcctttcatactgtgttgagtaacaccctagggtgtccattaaagttttgagttttacctagagcccagagccatgaatcaggattctgtctacatgattcgtgttttcattggtgtcaaaatacaaaagccaaagttctggctatgaattgttaacttggaagaaatactaactgccaccacttattaagtgcctactgtgtgccaggctctgaactaggtgcttcatatacattatcctaaattatctcaacatatgaggtaggtgttttaatttttattttatagaacttggtgtgtttgactgttaagctatggggctagagagagggtttgatcccaggtccctctgtgcttttgctgctgagccacacaacctctcatttcaaaaacactttcaaaatgctaacatattctaattcactctaggccaccaaaaactttaatactaatatctgatttgtaaatgacttaatgtatccttgaccctatcagctgaatttaatgaaatattcctctctgctgtgaaattttaccagtatagtatttggtctagtgacaggtgttctatttttttgagacgggtctcactctgtcacccaggctggagtgcagtggctcaatgcaacctctgccccccaagttcaagtgattctcctgcctcagcctcttgagtagctgggattacaggcacatgcaccacgcccggctgatttttttttgtatttttagtagacagggtttcaccatgttggccaggctggtcttgaactcctgacctcaggtgatccgcccgcctctgcctcccaaagtgctgggattacaggtgtaagccatcacacctggccctagtgacaggtttttatgggtacttttagatgatctaagaaatcatgtgcatatatctttcagatttctattttgggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccagggcctggttattgttcagacattccatggtgattcctgggaaggaagcttggctgctcagtttctgagtctggggtgagataatgttctgggaagggacatctgttctttggtgtaatctctcatggtgaaatctgctctgtacatcagacaattgcattgctaccaagtttcataccaaatatttgaaaggatggtattgaatctaaaaccaaatattagtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaaaaaaaaaa

SEQ ID NO: 17 - DNA, 3659 bp, homo sapiens
aactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattctcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtccagctactcgggaggctgaggcaggagaatggcatgaaccccaggctggagtgcagtggctcaatgcaacctctgccccccgggttcggtgattctcctgcctcagcctcttgagtggctgggattgcgggcacatgcaccacgcccggctgatttttttttgtatttttagtggacagggtttcaccatgttggccaggctggtcttgaactcctgacctcaggtgatccgcccgcctctgcctcccaaggtgctggattacaggtgtaagccatcacacctggccctagtgacaggtttttatgggtacttttagatgatctaagaaatcatgtgcatatatctttcagatttctattttggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccaggcctggttattgttcagacattccatggtgattcctgggaaggaagcttggctgctcagtttctgagtctggggtgagataatgttctggaaggacatctgttctttggtgtaatctctcatggtgaaatctgctctgtacatcagacaattgcattgctaccaagtttcataccaaatatttgaaaggatggtattgaatctaaaccaaatattaggtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaa

SEQ ID NO: 18 - DNA, 3659 bp, homo sapiens
aactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacagaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtccagctactcgggaggctgaggcaggagaatggcatgaaccccaggctggagtgcagtggctcaatgcaacctctgccccccgggttcggtgattctcctgcctcagcctcttgagtggctgggattgcgggcacatgcaccacgcccggctgatttttttttgtatttttagtggacagggtttcaccatgttggccaggctggtcttgaactcctgacctcaggtgatccgcccgcctctgcctcccaaggtgctggattacaggtgtaagccatcacacctggccctagtgacaggtttttatgggtacttttagatgatctaagaaatcatgtgcatatatctttcagatttctattttggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccaggcctggttattgttcagacattccatggtgattcctgggaaggaagcttggctgctcagtttctgagtctggggtgagataatgttctggaaggacatctgttctttggtgtaatctctcatggtgaaatctgctctgtacatcagacaattgcattgctaccaagtttcataccaaatatttgaaaggatggtattgaatctaaaccaaatattaggtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaa

SEQ ID NO: 19 - DNA, 3659 bp, homo sapiens
aactctattttaagaacctctcaaaacgaaacaagcaaatcatggagaagaatggaaataaccgaaagctgtgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtccagctactcgggaggctgaggcaggagaatggcatgaaccccaggctggagtgcagtggctcaatgcaacctctgccccccgggttcggtgattctcctgcctcagcctcttgagtggctgggattgcgggcacatgcaccacgcccggctgatttttttttgtatttttagtggacagggtttcaccatgttggccaggctggtcttgaactcctgacctcaggtgatccgcccgcctctgcctcccaaggtgctggattacaggtgtaagccatcacacctggccctagtgacaggtttttatgggtacttttagatgatctaagaaatcatgtgcatatatctttcagatttctattttggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccaggcctggttattgttcagacattccatggtgattcctgggaaggaagcttggctgctcagtttctgagtctggggtgagataatgttctggaaggacatctgttctttggtgtaatctctcatggtgaaatctgctctgtacatcagacaattgcattgctaccaagtttcataccaaatatttgaaaggatggtattgaatctaaaccaaatattaggtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaa

SEQ ID NO: 20 - DNA, 2304 bp, homo sapiens
attatttcaaaccacactagggtacagagctcgtgcttcgggatggaaacctatggttatctgcagagggagtcatgctttcaaggacctcatgaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactag

SEQ ID NO: 21 - DNA, 2174 bp, homo sapiens
cggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctat

SEQ ID NO: 22 - DNA, 1966 bp, Adeno-associated virus - 2
cggtccgaagcgcgcggaattcaaaggcctacgtcgacgaggggagatctgccgccctggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaactcgaggacaatcaagcttgcatgcctgcaggtcgactctaga

SEQ ID NO: 23 - DNA, 1876 bp, Adeno-associated virus - 2
cgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 24 - DNA, 4404 bp, deno-associated virus - 5
cgcgacaggggggagagtgccacactctcaagcaagggggttttgtaagcagtgatgtcataatgatgtaatgcttattgtcacgcgatagttaatgattaacagtcatgtgatgtgttttatccaataggaagaaagcgcgcgtatgagttctcgcgagacttccggggtataaaagaccgagtgaacgagcccgccgccattctttgctctggactgctagaggaccctcgctgccatggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaaataaagcgagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtcattcaatgaataacagcttacaacatctacaaaacctccttgcttgagagtgtggcactctcccccctgtcgcgcg

SEQ ID NO: 25 - DNA, 4393 bp, Adeno-associated virus - 8
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 26 - DNA, 3357 bp, Mus musculus
ccatcctggtctatagagagagttccagaacagccagggctacagataaacccatctggaaaaacaaagttgaatgacccaagaggggttctcagagggtggcgtgtgctccctggcaagcctatgacatggccggggcctgcctctctctgcctctgaccctcagtggctcccatgaactccttgcccaatggcatctttttcctgcgctccttgggttattccagtctcccctcagcattccttcctcagggcctcgctcttctctctgctccctccttgcacagctggctctgtccacctcagatgtcacagtgctctctcagaggaggaaggcaccatgtaccctctgtttcccaggtaagggttcaatttttaaaaatggttttttgtttgtttgtttgtttgtttgtttgtttgtttttcaagacagggctcctctgtgtagtcctaactgtcttgaaactccctctgtagaccaggtcgacctcgaactcttgaaacctgccacggaccacccagtcaggtatggaggtccctggaatgagcgtcctcgaagctaggtgggtaagggttcggcggtgacaaacagaaacaaacacagaggcagtttgaatctgagtgtattttgcagctctcaagcaggggattttatacataaaaaaaaaaaaaaaaaaaaaaccaaacattacatctcttagaaactatatccaatgaaacaatcacagataccaaccaaaaccattgggcagagtaaagcacaaaaatcatccaagcattacaactctgaaaccatgtattcagtgaatcacaaacagaacaggtaacatcattattaatataaatcaccaaaatataacaattctaaaaggatgtatccagtgggggctgtcgtccaaggctagtggcagatttccaggagcaggttagtaaatcttaaccactgaactaactctccagccccatggtcaattattatttagcatctagtgcctaatttttttttataaatcttcactatgtaatttaaaactattttaattcttcctaattaaggctttctttaccatataccaaaattcacctccaatgacacacgcgtagccatatgaaattttattgttgggaaaatttgtacctatcataatagttttgtaaatgatttaaaaagcaaagtgttagccgggcgtggtggcacacgcctttaatccctgcactcgggaggcaggggcaggaggatttctgagtttgaggccagcctggtctacagagtgagttccaggacagccagggctacacagagaaaccctgtctcgaaccccccaccccccaaaaaaagcaaagtgttggtttccttggggataaagtcatgttagtggcccatctctaggcccatctcacccattattctcgcttaagatcttggcctaggctaccaggaacatgtaaataagaaaaggaataagagaaaacaaaacagagagattgccatgagaactacggctcaatattttttctctccggcgaagagttccacaaccatctccaggaggcctccacgttttgaggtcaatggcctcagtctgtggaacttgtcacacagatcttactggaggtggtgtggcagaaacccattccttttagtgtcttgggctaaaagtaaaaggcccagaggaggcctttgctcatctgaccatgctgacaaggaacacgggtgccaggacagaggctggaccccaggaacaccttaaacacttcttcccttctccgccccctagagcaggctcccctcaccagcctgggcagaaatgggggaagatggagtgaagccatactggctactccagaatcaacagagggagccgggggcaatactggagaagctggtctccccccaggggcaatcctggcacctcccaggcagaagaggaaacttccacagtgcatctcacttccatgaatcccctcctcggactctgaggtccttggtcacagctgaggtgcaaaaggctcctgtcatattgtgtcctgctctggtctgccttccacagcttgggggccacctagcccacctctccctagggatgagagcagccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 27 - DNA, 310 bp, homo sapiens
ctcctcccccacacagagtccttagctcctgggactgagagctgaggttcagaggggccagggaggcggtgggggggtggggggtgggggaagggtaataatggctagctccaaaacagccccggcagctgtccctgtcacagagaggagactgtgtgacggtacgtgtctgtctgcctggatgtggcagcgcatgtgtgggagagtgtgtgtttgtgtgcccctagctccaagtccaagtgcttattatgtctgagtgggggcctgtgtgtgtctgcacatgtgtctgtctgtctctgggaacacgcag

SEQ ID NO: 28 - DNA, 8046 bp, Artificial Sequence
  recombinant plasmid.
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctcgagtcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 29 - DNA, 6389 bp, Artificial Sequence
  recombinant plasmid
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttctagacagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcatctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgcgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcatcgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

(19) United States
(12) Patent Application Publication
(10) Pub. No.: US 2015/0045416 A1
(43) Pub. Date: Feb. 12, 2015

(54) Methods and Compositions for Gene Delivery

(71) Applicant: HADASIT MEDICAL RESEARCH SERVICES & DEVELOPMENT LTD., Jerusalem (IL)

(72) Inventors: Stella Mitrani-Rosenbaum, Jerusalem (IL); Avizohar Argov, Jerusalem (IL)

(21) Appl. No.: 14/370,594

(22) PCT Filed: Jan. 3, 2013
(86) PCT No.: PCT/IL2013/050014

§ 371 Date: Jul. 3, 2014

Related U.S. Application Data
(60) Provisional application No. 61/631,456, filed on Jan. 5, 2012.

(51) Int. Cl. C12N 15/86; A61K 45/06; A61K 31/713

(52) U.S. Cl. CPC C12N 15/86; A61K 31/713; A61K 45/06
USPC 514/44 R; 435/320.1; 435/369

(57) ABSTRACT

Disclosed herein are AAV-based viral vectors encoding GNE from muscle-specific and non-muscle specific promoters, and the use of same in treating myopathies associated with altered GNE function.

### FIELD

AAV-based viral vectors encoding GNE, and the use of same in treating myopathies associated with altered GNE function, are provided.

### BACKGROUND

GNE myopathy, a recessive adult onset myopathy variously known as hereditary inclusion body myopathy (HIBM) (Askanas and Engel, 1998), quadriceps sparing myopathy (Argov and Yarom, 1984), and distal myopathy with rimmed vacuoles (DMRV, Nonaka's disease) (Nonaka et al., 1981), is caused by mutations in the UDP-N-acetylglucosamine 2 epimerase/N-acetylmannosamine kinase-encoding gene (GNE), the key enzyme in the biosynthesis pathway of sialic acid. The condition has a worldwide distribution, with most patients being compound heterozygotes, carrying mutations either at the epimerase domain, or at the kinase domain, or one in each domain of the GNE gene.

The process by which mutations in GNE lead to muscle disease is not understood. A transgenic mouse model generated on a GNE^{-/-} background and over-expressing a frequent mutation in Japanese patients, the D176V GNE missense mutation occurring in the epimerase domain of the enzyme, has been found to be a relevant model for GNE myopathy (Malicdan et al., 2007; Maclidan et al., 2009).

U.S. 2009/0298112 discusses methods of treating GNE myopathy is a subject comprising identifying a subject in need thereof, and administering to the subject a compound, or a pharmaceutically acceptable salt, ester, amide, glycol, peptidyl, or prodrug thereof, wherein the compound is a compound that is biosynthesized in a wild type individual along a biochemical pathway between glucose and sialic acid, inclusive. Also discussed therein are vectors comprising a nucleic acid sequence that encodes a polypeptide having at least 80% sequence identity to a GNE isoform 1 sequence, recombinant cells comprising these vectors, and recombinant animals comprising the cells. In addition, methods of identifying a compound having a therapeutic effect for GNE myopathy are described.

Recently, a gene therapy treatment has been reported for a single GNE myopathy patient, by injection of the GNE gene delivered via liposomes (Nemunaitis et al., 2011). Although it has been shown that wt GNE mRNA was expressed in the patient's quadriceps, this was assayed only 72 hours after injection, and its efficacy could not be properly evaluated because of the severity of the patient's condition prior to the injection.

### SUMMARY

Disclosed herein are AAV-based viral vectors encoding GNE from muscle-specific and non-muscle specific promoters, and the use of same in treating myopathies associated with altered GNE function. While the use of AAV-based vectors is known in the art, their use in treating myopathies associated with altered GNE function has not been heretofore considered, to the inventors' knowledge. The present disclosure demonstrates the considerable efficacy of such vectors in treating these types of myopathies.

### BRIEF DESCRIPTION OF THE FIGURES

FIG. 1. GFP expression in cells transduced with AAV/GNE. Percentage of murine C2C12 (top panel) and human GNE myopathy muscle cells (bottom panel) expressing GFP at different time points, after transduction with 1×10⁵ AAV8/GNE-IRES-GFP viral vectors per 2×10⁵ cells.

FIG. 2. Human GNE mRNA expression in cells transduced with AAV/hGNE. Top Panel: Murine C2C12 cells were transduced with AAV8/hGNE-IRES-GFP viral vectors and sorted for GFP expression 8 days after transduction for analysis of hGNE mRNA expression by RT/PCR with primers specific for human GNE versus mouse GNE. Bottom Panel: the expression of human wild-type GNE mRNA was analysed in muscle cell cultures from GNE myopathy patients carrying the M712T mutation 8 days after transduction with AAV8/hGNE-IRES-GFP viral vectors (1×10⁵ viral vectors per 2×10⁵ cells), by RT-PCR using the ARMS technique. As controls, normal or GNE myopathy cells were assayed with the primers set detecting only the wild-type (Wt) or the mutated (Mut) cDNA, M denotes molecular weight standards.

FIG. 3. Weight and grip force of mice injected with AAV vectors. Mice were injected intravenously with either 8.5×10¹¹ vg/mouse AAV8/hGNE-IRES-GFP (hGNE) or AAV8/luciferase-IRES-GFP (Lluc), with 2.4×10¹² vg/mouse AAV8/luciferase-IRES-GFP vector (Hluc) or with PBS. Weight (top panel) and grip force (bottom panel) were monitored at different time points after injection.

FIGS. 4A and 4B. Luciferase activity in AAV/Luciferase-transduced mice. (4A) Representative in vivo bioluminescence images obtained at different time points after injection of AAV/luciferase-IRES-GFP at 8.5×10¹¹ vg/mouse (Lluc), or 2.4×10¹² vg/mouse (Hluc), in a IVICS Kinetic system (Caliper Life Sciences, Hopkinton, Mass.). Luminescence appears as patches on the grayscale images. (4B)) Luciferase activity quantification expressed as average radiance (p/sec/cm²/sr).

FIG. 5. hGNE mRNA expression in muscles of mice injected with AAV vectors. Quantitative expression of hGNE mrRNA was analyzed by real-time PGR in different muscles of mice at different time points after transduction with either 8.5×10¹¹ vg/mouse AAV8/hGNE-IRES-GFP (hGNE) or AAV/8luciferase-IRES-GFP (Lluc), 2.4×10¹² vg/mouse AAV8/luciferase-IRES-GFP vector (Hluc) or with PBS. Relative Quantitative expression (RQ) or fold expression for each sample is defined as the ratio between the normalized hGNE and mGNE (hGNE/mGNE) values, and is relative to the highest value detected with control murine tissue (either the tissue of mice injected with AAV8-Luciferase-IRES-eGFP at high or low dose, or the tissue of mice injected with PBS, as appropriate), which was set as as RQ=1.

FIG. 6. hGNE mRNA expression in tissues of mice injected with AAV vectors. Quantitative expression of hGNE mRNA was analyzed by real-time PCR in different tissues of mice at different time points after transduction with either 8.5×10¹¹ vg/mouse AAV8/hGNE-IRES-GFP (hGNE) or AAV8/luciferase-IRES-GFP (Lluc), 2.4×10¹² vg/mouse AAV8/luciferase-IRES-GFP vector (Hluc) or with PBS. Relative Quantitative expression (RQ) or fold expression was defined as described for FIG. 5.

FIGS. 7A and 7B. (7A) Plasmids used for AAV-derived vector production. To produce AAV8 viral vectors, HEK293 cells were triple-transfected with pHelper, pAAV8, and either pCMV-hGNE-IRES-GFP or pCMV-Luc-IRES-GFP plasmids. pCMV-hGNE-IRES-GFP was generated by replacing the luciferase gene from pCMV-Luc-IRES-GFP by the hGNE cDNA at BamHI-EcoRI sites. (7B) Magnified diagram of pCMV-hGNE-IRES-GFP.

FIG. 8. AAV8/hGNE copy number in various mouse tissues after AAV/hGNE injection. Various tissues and muscles were analysed for viral copy number at different time points after injection of AAV8/hGNE-IRES-GFP viral vectors, by real-time quantitative PCR. Two tissue DNA measurements were performed, of 100 ng and 10 ng respectively, and the average was calculated against a standard curve obtained with the plasmid. For calculation of vg per cell, 1 ng tissue was considered equivalent to 150 genome copies.

FIG. 9. Histology of mouse tissues 45 days after transduction with AAV8 vectors. Mice paraffin tissue sections and muscle frozen sections of mice 45 days after injection with the various AAV8 vectors were stained by hematoxilin and eosin. All pictures were captured at ×20 magnification, except liver sections (×40) and kidney sections (×10).

FIG. 10. Histology of mouse tissues 178 days after transduction with AAV vectors. Mice paraffin tissue sections (5μ) and muscle frozen sections (8μ) after 45 days of injection with the various AAV8 vectors were stained by hematoxilin and eosin. All pictures were captured at ×20 magnification, except liver sections (×40) and kidney sections (×10).

FIG. 11. Histology of additional mouse tissues 45 and 178 days after transduction with AAV8 vectors. See description of FIG. 10 above.

FIG. 12. IP-10 level in serum of AAV-injected mice. IP-10 levels (pg/ml) were determined by ELISA on sera from mice injected with 8.5.10¹¹ vg/mouse AAV8/hGNE-IRES-GFP (hGNE) or AAV8/luciferase-IRES-GFP (Lluc), 2.4.10¹² vg/mouse AAV8/luciferase-IRES-GFP vector (Hluc) or with PBS, at various time points. Measurements were taken for 4 mice in each group until day 43 and for 3 mice until day 92.

FIG. 13. Schematic diagram of AAV-MCK-GNE, an AAV-based vector expressing hGNE under tbe control of a muscle-specific promoter.

FIG. 14. Expression of human GNE mRNA in AAV/hGNE injected mice in various tissues. Mice were injected with 1×10¹² viral vector genomes in the tail vein. Each column represents one mouse. The y axis scale represents the fold expression-relative quantity-(RQ) of hGNE (specific Taqman probe) mRNA relative to the expression measured in PBS injected mouse in the same organ (RQ=1). All values were normalized to GNE expression of the relevant endogeneous mouse tissue. “MCK/GNE” and “CMV/GNE” denote the AAV8 viral vector containing the wild type human GNE driven by the MCK promoter or the CMV promoter, respectively.

### DETAILED DESCRIPTION

Described herein is an adeno-associated virus (AAV)-based viral vector, comprising a nucleotide sequence that encodes a UDP-N acetylglucosamine 2 epimerase/N-acetylmannosamine kinase (GNE) functionally linked to a promoter. In certain embodiments, the AAV-based vectors comprise an AAV packaging signal. In more specific embodiments, the AAV-based vectors comprise an AAV packaging signal and do not contain any the rep and cap genes, or in other embodiments, if fragments of the rep and cap genes are present, said fragments are too small to be functional. In other embodiments, the AAV-based vectors comprise both an AAV packaging signal and the rep and cap genes.

The GNE gene has GenBank Gene ID No. 10020. Representative sequences include GenBank Accession Nos. NM_{—}001128227, NM_{—}001190383, NM_{—}001190384, NM_{—}001190388, NM_{—}005476, AY531127, AY531128, AY531126, AK312539, and EU093084, all accessed on Dec. 25, 2012 (SEQ ID NOs 12-21, respectively). In certain embodiments, the gene is selected from transcript variants 1, 2, 3, 4, and 5 of GNE, each of which represents a separate embodiment.

In certain embodiments, the GNE expressed by the vector is a human GNE. In more specific embodiments, the gene is selected from transcript variants 1, 2, 3, 4, and 5 of human GNE, each of which represents a separate embodiment. The skilled artisan will appreciate in light of the present disclosure that various GNE proteins that are functional in human muscle tissue, such as mutants of human GNE, non-human GNE proteins, and mutants of same, and thus genes encoding such forms of GNE can also be used. Genes encoding metabolically-functional GNE proteins are generally preferred. In certain embodiments, a fully-functional GNE is used. “Fully-functional GNE” in this context refers to a GNE gene that exhibits an activity in the sialic acid biosynthesis that is at least equivalent to wild-type human GNE. Methods of assaying GNE catalytic activity are known in the art, and are described, inter alia, in Keppler et al 1999.

AAV-based vectors are produced inter alia by Amsterdam Molecular Therapeutics B.V. (NL), Microbix Biosystems Inc. (Mississauga, Ontario, Canada), NanoCor Therapeutics, Inc (Chapel Hill, N.C., USA), and Vector Gene Technology Company, Ltd (Beijing, China). Partial and complete AAV sequences and the production and use of AAV-based vectors are described, inter alia, in GenBank Accession numbers HC000068 (SEQ ID NO 22), HC000057, HC000061, HC000044 (SEQ ID NO 23), HC000041, HC000039, HC000059, HC000046, HC000042, HC000040, HC000038, Y18065 (SEQ ID NO: 24), NC_{—}006261 (SEQ ID NO: 25), all accessed on Dec. 25, 2012, and in U.S. Patent Publications 2011/0136227, 2012/0253018, 2012/0232133, 2012/0220648, 2012/0164106, 2012/0028357, 2011/0236353, 2010/0260800, 2010/0227407, 2010/0310601, 2010/0278791, and U.S. Pat. Nos. 8,318,687, 8,298,818, 8,273,344, 7,456,015, 7,094,604, and 6,670,176, all of which are incorporated by reference, as well as in Gadalla et al, which is incorporated by reference. The general safety and efficacy of AAV has been well documented, including clinical trials using AAV platforms (Carter, 2005; Maguire et al., 2008; Park et al., 2008; Nathwani et al, 2011; and Hu et al 2010, all of which are incorporated by reference).

In certain embodiments, the AAV-based vector is a recombinant vector. Alternatively or in addition, the vector is a vector that was created by introduction of the GNE gene into an AAV virus or vector.

In certain embodiments, the AAV/like vectors are selected from AAV serotypes 1, 2, 3, 4, 5, 6, 7, 8, 9, 10, and 11, each of which represents a separate embodiment. For example, the AAV vector may contain the capsid sequence of an AAV8 vector. While AAV8 is utilized herein, the skilled artisan will appreciate, in light of the present disclosure, that various AAV vectors are suitable for in-vivo GNE expression in the context of the described compositions and methods. The availability of multiple AAV serotypes allows efficient targeting to many tissues of interest (Gao et al, 2002; McCarty, 2008; U.S. Patent Publications 2008/075737, 2008/0050343, 2007/0036760, 2005/0014262, 2004/0052764, 2003/0228282, 2003/0013189, 2003/0032613, and 2002/0019050, each incorporated herein by reference). Alternatively or in addition, the vectors are self-complementary (sc) AAV vectors, which are described, for example, in U.S. Patent Publications 2007/01110724 and 2004/0029106, and U.S. Pat. Nos. 7,465,583 and 7,186,699 (all of which are incorporated by reference). Additional vectors are described in U.S. Patent Publication U.S. 2011/0301226, which is incorporated by reference.

In other embodiments, recombinant AAV vectors can be produced by a triple transfection method, for example using: (i) scAAV.GNE, for example hGNE, (ii) a rep-cap AAV helper plasmid encoding the rep and cap transcripts, and (iii) an adenovirus helper plasmid (pAdhelper) expressing adenovirus E2A, E4 ORF6, and VA I/II RNA genes.

In yet other embodiments, the plasmid used to produce the genome of the described AAV vector contains capsid signal sequences taken from one AAV serotype (for example selected from AAV serotypes 1, 2, 3, 4, 5, 6, 7, 8, 9, 10, and 11) and packaging sequences from a different serotype (for example selected from AAV serotypes 1, 2, 3, 4, 5, 6, 7, 8, 9, 10, and 11, an example of which is an AAV 2/8 vector, which contains the capsid sequence of an AAV8 vector and the signal sequence from an AAV2 vector. The signal sequence present in the AAV vector is not believed to significantly affect the in-vivo efficacy for the purposes described herein.

The term “functionally linked to a promoter”, as used herein, indicates that the GNE gene is expressed under control of the promoter. In other words, the promoter directs expression of the GNE gene. In various embodiments, the vector described herein may or may not contain an internal ribosome entry site (IRES) for the GNE open reading frame.

The nucleotide sequence that encodes GNE can be, in non-limiting embodiments, a cDNA, such as a naturally-occurring cDNA or a modified cDNA sequence. Those skilled in the art will recognize, in light of the present disclosure that other suitable types of nucleotide sequence can also be utilized.

The promoter used to express the nucleotide sequence encoding GNE is, in certain embodiments, a muscle-specific promoter. In other embodiments, it is a non-muscle-specific promoter. “Muscle-specific promoter” in this context refers to a promoter that, in the context of its surrounding sequence that is included in the vector, provides at least 5-fold higher expression in a muscle cell than in a reference cell such as an epithelial cell. In alternative embodiments, the expression in muscle cells is at least 7-fold, at least 10-fold, at least 15-fold, or at least 20-fold greater than the reference cell.

A non-limiting example of a muscle-specific promoter is the muscle creatine kinase (CKM) promoter. Non-limiting examples of suitable muscle creatine kinase promoters are human muscle creatine kinase promoters and truncated murine muscle creatine kinase (tMCK) promoters) (Wang B et al, Construction and analysis of compact muscle-specific promoters for AAV vectors. Gene Ther. 2008 Nov.; 15(22):1489-99) (representative GenBank Accession No. AF188002; SEQ ID NO 26). Human muscle creatine kinase has the Gene ID No. 1158 (representative GenBank Accession No. NC_{—}000019.9, accessed on Dec. 26, 2012). Other examples of muscle-specific promoters include myosin light chain (MLC) promoters, for example MLC2 (Gene ID No. 4633; representative GenBank Accession No. NG_{—}007554.1, accessed on Dec. 26, 2012); myosin heavy chain (MHC) promoters, for example alpha-MHC (Gene ID No. 4624; representative GenBank Accession No. NG_{—}023444.1, accessed on Dec. 26, 2012); desmin promoters (Gene ID No. 1674; representative GenBank Accession No. NG_{—}008043.1, accessed on Dec. 26, 2012); cardiac troponin C promoters (Gene ID No. 7134; representative GenBank Accession No. NG_{—}008963.1, accessed on Dec. 26, 2012); troponin I promoters (Gene ID Nos. 7135, 7136, and 7137: representative GenBank Accession Nos. NG_{—}016649.1, NG_{—}011621.1, and NG_{—}007866.2, accessed on Dec. 26, 2012); myoD gene family promoters (Weintraub et al., Science, 251, 761 (1991); Gene ID No. 4654; representative GenBank Accession No. NM_{—}002478, accessed on Dec. 26, 2012); actin alpha promoters (Gene ID Nos. 58, 59, and 70; representative GenBank Accession Nos. NG_{—}006672.1, NG_{—}011541.1, and NG_{—}007553.1, accessed on Dec. 26, 2012); actin beta promoters (Gene ID No. 60; representative GenBank Accession No. NG_{—}007992.1, accessed on Dec. 26, 2012); actin gamma promoters (Gene ID No. 71 and 72; representative GenBank Accession No. NG_{—}011433.1 and NM_{—}001199893, accessed on Dec. 26, 2012); muscle-specific promoters residing within intron 1 of the ocular form of Pitx3 (Gene ID No. 5309) (Coulon et al; the muscle-specific promoter corresponds to residues 11219-11527 of representative GenBank Accession No. NG_{—}008147, accessed on Dec. 26, 2012; these residues are provided in the accompanying sequence ID listing as SEQ ID NO 27); and the promoters described in U.S. Patent Publication U.S. 2003/0157064, which is incorporated herein by reference.

In certain embodiments, the described viral vectors may be modified with a modification designed to reduce their immunogenicity. A non-limiting example of such a modification is a mutation that reduces the number of surface-exposed tyrosine residues. It will be appreciated by those skilled in the art in light of the present disclosure that improving the capacity of AAV to avoid an immunogenic response could ensure an effective reuse of the viral vectors if needed. Recent promising studies relate to modulating the viral capsid structure to obtain more specific cell targeted transduction (Markusic et al., 2010), or by immunosuppression (McIntosh et al., 2011). It should be noted that the immune response to the normal transgene GNE itself is of much less concern in this specific case of GNE myopathy, since the mutated GNE protein is expressed in the patients at normal levels (Krause et al., 2007). It will be also appreciated that a strong immunologic response of the organism to a protein with only one single nucleotide change is highly improbable.

Also provided is a host cell comprising a viral vector as described herein.

Additionally, a pharmaceutical composition comprising a viral vector as described herein is provided.

Also provided herein is a method of treating a subject suffering from a myopathy associated with a deficient GNE function, comprising the step of administering a pharmaceutical composition comprising a viral vector as described herein. As provided herein, a single administration of a described pharmaceutical composition confers lasting expression, namely stable expression for at least six months, of GNE. The viral vector may have any of the attributes described herein, each of which represents a separate embodiment.

Use of a viral vector as described herein, in the preparation of a medicament for treating a myopathy associated with a deficient GNE function, is also provided herein. The viral vector may have any of the attributes described herein, each of which represents a separate embodiment.

In certain embodiments, a pharmaceutical composition described herein, or one used in a method thereof, is indicated for treating a myopathy associated with deficient GNE function. Specific examples of such myopathies include hereditary inclusion body myopathy (HIBM), quadriceps sparing myopathy, distal myopathy with rimmed vacuoles (DMRV) and Nonaka's disease. The viral vector may have any of the attributes described herein, each of which represents a separate embodiment.

Some embodiments relate to treating an established myopathy. Compositions described herein were surprisingly found to have significant efficacy in treating established myopathies. “Established myopathy” in this context refers to a symptomatic myopathy. Alternatively, the term may refer to a subject that presents with a symptomatic myopathy.

In some embodiments, the described pharmaceutical compositions are indicated for systemic administration. One non-limiting example of systemic administration is intravenous injection. Another embodiment is intraarterial administration. The compositions tested herein were shown to direct expression of GNE in muscle tissue, even when administered systemically.

In other embodiments, locoregional administration is used. In more specific embodiments, the locoregional administration is selected from intravenous administration in an affected muscle and intra-arterial administration in the vicinity of an affected muscle. In still more specific embodiments, intravenous or intra-arterial administration is performed on a a blood vessel in the vicinity of a muscle in an affected limb, for example an arm, leg, finger, or toe, in conjunction with restriction of the venous circulation of the treated limb. Methods of restricting the venous circulation of a limb include tourniquets and other devices capable of compressing a vein, as well as physical compression performed by a health care profession or the patient.

In other embodiments, the pharmaceutical compositions are indicated for administration together with immunosuppressive therapy. In this regard, “together with immunosuppressive therapy” refers, in some embodiments, to administration in such a manner that an immune response to the vector is blunted. Thus, the immunosuppressive therapy need not be administered at exactly the same time as the vector, provided that immunosuppression is achieved during the time window when an immune response to the vector would be mounted, typically within 3-14 days of administration of the vector; for example up to 3-14 days after administration of the vector or, alternatively, up 3-14 days before administration of the vector.

Also provided herein is a method of producing an AAV-GNE viral vector, comprising the step of introducing, into a host cell that expresses the E1A and E1B proteins, a first plasmid that comprises the E2A, E4 and VA RNA regions of an adenovirus; a second plasmid that comprises a GNE gene bounded by AAV inverted terminal repeats; and a third plasmid that comprises the AAV rep and capsid genes without the AAV inverted terminal repeats, and incubating such cell under conditions that enable expression of the genes contained in the plasmids.

Wherever alternatives for single features such as the vector subtype, GNE gene, promoter, etc. are laid out herein as “embodiments”, it is to be understood that such alternatives may be combined freely to form discrete embodiments of the entire formulation provided herein.

The invention is further illustrated by the following examples and the figures, from which further embodiments and advantages can be drawn. These examples are meant to illustrate the invention but not to limit its scope.

### Experimental Details

### Materials and Experimental Methods

### GNE Cloning and Virus Production

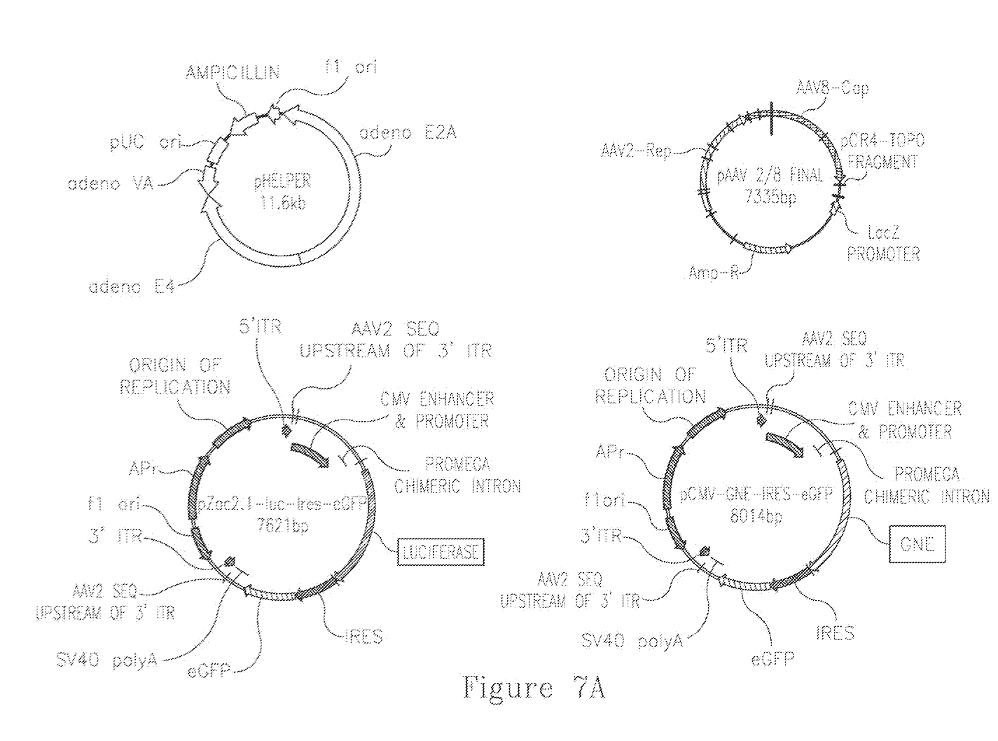

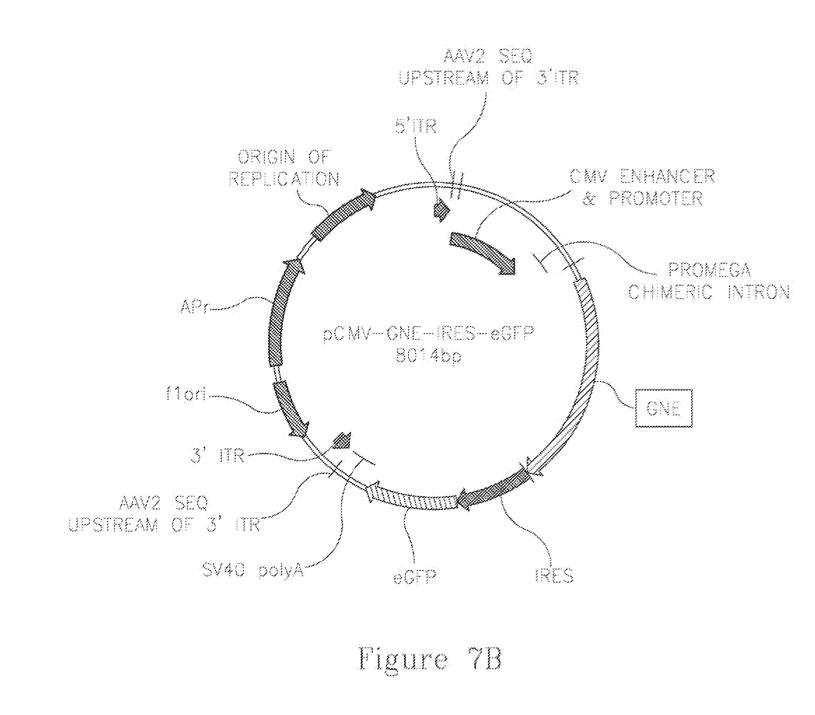

To produce AAV8 viral vectors carrying the human GNE gene (AAV8-hGNE), GNE cDNA was generated by PCR from the previously described N-terminal 3XFLAG-CMV-10 GNE vector (Amsili et al., 2008) and subsequently subcloned it into the pCMV-Luciferase-eGFP vector (pZac2.1-luc-IRES-eGFP, supplied by Penn Vector Core at University of Pennsylvania) by replacing the luciferase gene at EcoRI/BamHI sites (FIG. 7). Small-scale virus preparations for in vitro studies were produced by triple transfection into HEK 293 cells (Matsushita et al., 1998). The three plasmids used (FIG. 7) were the newly-generated pCMV-GNE-IRES-eGFP (SEQ ID NO: 28; the GNE cDNA spans nucleotides 1264-3475) or the original pCMV-luciferase-IRES-eGFP, the pRepCapAAV2/8 plasmid (AAV2/8 denotes that the plasmid has packaging signal sequences taken from the AAV2 sequence and capsid sequences from AAV8 provided by Penn Vector Core at University of Pennsylvania, and the pHelper plasmid from Stratagene. Virus was harvested after 72 hours by freeze/thaw cycles, followed by centrifugation. The titers of the viral vectors produced were assessed by the percentage of HEK293 cells expressing GFP 72 hours after transduction. Large-scale-purified pCMV-GNE-IRES-GFP and pCMV-Luciferase-IRES-eGFP viral vectors used for mice intravenous injection were produced and titrated by viral genome (vg) determination at the Penn Vector Core facility at the University of Pennsylvania.

### Cell Cultures

HEK293 and C2C12 cells were maintained in DMEM supplemented with 10% FCS penicillin/streptomycin and glutamine (Biological Industries, Beit Haemek, Israel). GNE myopathy-derived muscle cells were cultured as described by Lochmuller et al., 1999.

Cells were seeded and transduced in 6-well plates and harvested for analysis at different time points, GFP expression was analyzed by flow cytometry (FACSCalibur™ BD).

### Animal Procedures and Staining

8.5×10¹¹ vg or 2.4×10¹² vg of the viral vector in 250 microliters of PBS, or PBS, was injected into the tail vein of 5-6 week-old C57BL/6 mice. Mice were monitored for general behavior, and for weight and grip force using an Electronic Grip Strength Meter.

Mice were sacrificed at different time points and tissues specimens immediately processed for histology and RNA analysis (snap frozen and stored in liquid nitrogen until further processing). Different muscles were processed for frozen section histological analysis by snap-freezing in liquid nitrogen-cooled isopentane and were stored at −80° C.

Histological sections were stained for hematoxilin and eosin by standard procedures.

GNE mRNA Expression and Determination of Copy Number

Total RNA was extracted from cells and tissues at different time points with Tri-Reagent (Sigma, St. Louis, Mo. USA) according to the manufacturer's protocol. The Tri-Reagent samples containing the non-RNA sample fractions were stored at −80° C. for further DNA processing. After DNAse Invitrogen) treatment of RNA samples, RNA was reverse transcribed using random hexamer primers (Roche, Germany) by the Superscript® III reverse transcriptase enzyme (Invitrogen) according to the manufacturer's protocol. The cDNA products were amplified by PGR. Human GNE-specific primers, which do not detect the endogenous murine GNE, were used to detect the human GNE cDNA transgene expression in C2C12 murine cells. The GFP-positive and GFP-negative C2C12 populations were analyzed separately. The primers used were:

forward: 1131F-
(SEQ ID NO: 1)
5′-GGAAATGCTGTTCCAAGG-3′;
and
reverse: 1603R-
(SEQ ID NO: 2)
5′-GCACAGTTGCCATCATTGTC-3′.

A 470-bp product was obtained.

To detect human GNE cDNA transgene expression in GNE myopathy cells carrying the M712T mutation in GNE, the ARMS (amplification refactory mutation analysis: [Little, 1995]) technique, which can differentiate between the wild-type and mutated cDNA, was used. The primers used were:

ARMS F-5′-TGGAAGGCATGTCAGTGCCAAAAGATGAGG-3′ (SEQ ID NO: 3), which is common to both sequences and thus can be used for detection of both:

wt-R-5′-GTAGATCCTGCGTGTTGTGTAGTCCAGAACAA-3′ (SEG ID NO: 4), which can detect only the wild-type sequence; and

Mut-R 5′ GTAGATCCTGCGTGTTGTGTAGTCCAGAACAG 3′ (SEQ ID NO: 5), which can detect only the mutated M712T sequence.

The amplified product was 335 bp long.

To detect human GNE cDNA transgene expression in mouse tissues, quantitative real-time PCR was used with a TaqMan® set containing primers and a probe specifically designed for detection of human GNE cDNA (human GNE exons 7-8):

hF-5′ TCTTGGCGGGACGAACCTCCGA 3′ (SEQ ID NO: 6);

hR 5′ ACACACATCTGTAGGATTAAAT 340 (SEQ ID NO: 7); and

hGNEprobe-6-carboxyfluorescein(FAM™)-TTGCAATAGTCAGCATGAAG-Black Hole Quencher® (BHQ®) (SEQ ID NO: 8).

Endogenous mouse GNE expression was simultaneously measured in the same samples with a TaqMan® set containing primers and a probe specifically designed for mouse detection of endogenous GNE cDNA, in the very same region (mouse GNE exons 7-8):

mF-
(SEQ ID NO: 9)
5′ TCTTGGCGGGACAAACCTGAGG 3′;
mR-
(SEQ ID NO: 10)
5′ ACACACATCTGCAGGATTAAAC 3′;
and
mGNEprobe:
(SEQ ID NO: 11)
FAM-TGGCAATAGTTAGCATGAAG-BQ.

The analysis was performed in an ABI Prism 7500 real-time PCR system (Applied Biosystems, UK).

Relative Quantification (RQ) of hGNE expression in each sample was relative to the highest value detected with control murine tissue (RQ=1, either the tissue of mice injected with AAV8-Luciferase-IRES-eGFP at high or low dose, or the tissue of mice injected with PBS, as appropriate). All measurements were performed in duplicate and normalized relative to mouse HPRT expression (Mm00446968_ml, Applied Biosystems, UK).

Transgene copy number was determined by ABI Prism 7500 real-time PCR system. (Applied Biosystems, UK), using the same Taqman® human GNE specific probe set, since the transgene is hGNE cDNA. DNA was extracted using Tri-Reagent preparations. Duplicate samples of DNA of different tissues were analysed simultaneously and compared with a standard curve of determined quantities of the pCMV-hGNE-IRES-GFP plasmid.

### Luciferase Activity

Luciferase activity was analyzed in vivo in mice injected with pCMV-Luciferase-IRES-eGFP carrying viral vectors. Animals were dosed with 165 mg/kg body weight of Beetle Luciferin (Promega), intraperitoneally (i.p) in 0.5 ml of stock solution, 5 minutes prior to imaging. Imaging was performed in an IVIS Kinetic system (Perkin Elmer).

### IP-10 Measurements

Mice sera were analyzed for the quantitative determination of mouse interferon gamma inducible protein (IP-10) level by enzyme-linked immunosorbent assay (ELISA) using the Mouse CXCL10/IP-10/CRG-2 Quantikine® Immunoassay kit (R&D Systems), according to the manufacturer's instructions.

hGNE mRNA Expression and Biodistribution Determination

Mice in each group were sacrificed at day 45, 94 or 178 after transduction, and their tissues were analyzed by histology (H&E) for inflammation and tissue damage, and by real-time PCR for viral copy number and human GNE mRNA expression.

### Results

### EXAMPLE 1

### AAV/hGNE Transduction of Muscle Cells

### Transduction of C2C12 Cells

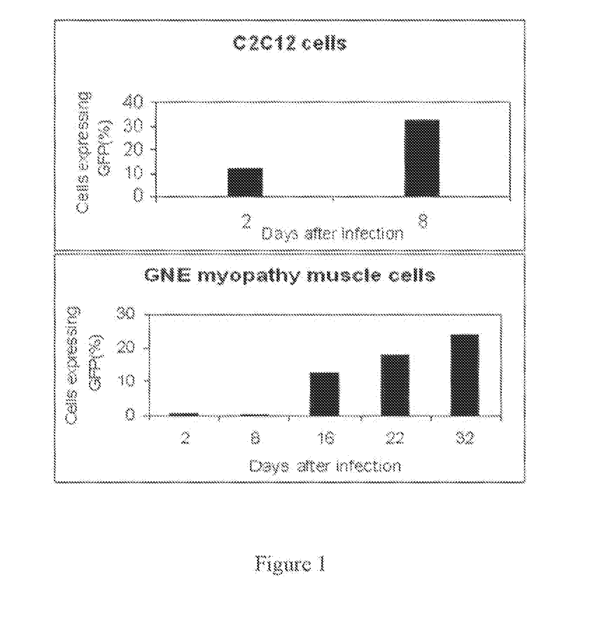

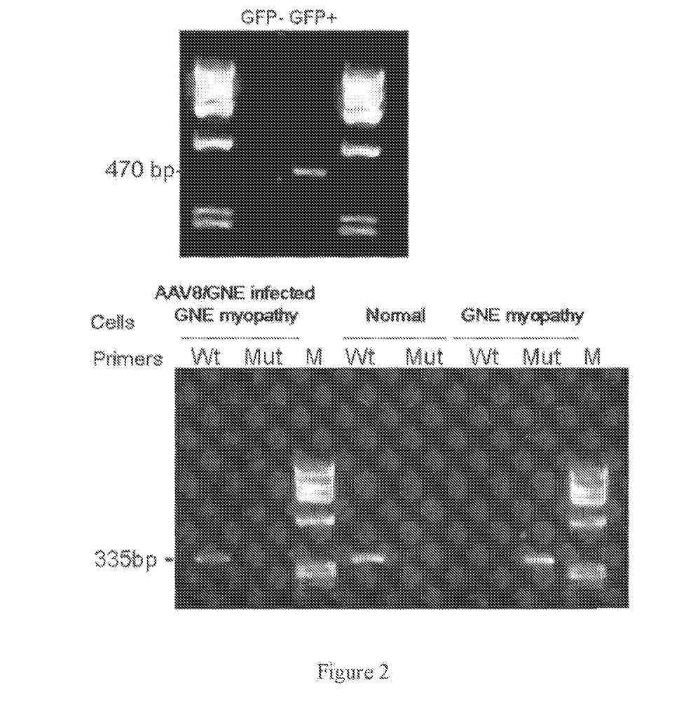

Human GNE cDNA was subcloned into a vector containing AAV packaging signals and GFP (FIG. 7A), and AAV/8/hGNE-IRES-GFP viral vector preparations were produced by triple transfection of HEK293 cells. In order to evaluate the potential of AAV8/hGNE-IRES-GFP viral vectors to transduce muscle cells, the C2C12 murine muscle cell line was transduced with the viral vectors (1×10⁶ infectious particles/ml) and analyzed for expression. GFP was detected in 12% and 30% of the cells after 2 and 3 days, respectively, after transduction (FIG. 1A). Transduced cells were sorted for GFP expression and analyzed for the presence of specific human GNE mRNA. Indeed, the GFP-positive cells expressed human GNE mRNA, while the GFP negative fraction did not (FIG. 2A).

### Transduction of Human Primary Muscle Cells

Subsequently, human primary muscle cell cultures derived from biopsies of GNE myopathy patients homozygous for the M712T mutation were transduced with the viral vectors (10⁵ infectious particles/ml) and analyzed for GFP expression and for the presence of normal human GNE mRNA, up to 32 days after transduction (a time point at which the muscle cells became naturally senescent). GFP was detected in a very low percentage of cells initially, but at 8-days post transduction, expression increased, reaching approximately 22% of the cells (FIG. 1B). Normal human exogenous GNE mRNA was also specifically detected in these cells, using the ARMS technique, which can distinguish between the mutated, endogenous and exogenous, wild-type GNE. While the untransduced GNE myopathy cells expressed only the mutated GNE mRNA, the transduced cells expressed wild-type hGNE mRNA (FIG. 2B).

These findings demonstrate that engineered AAV8 viral vectors carrying human wild-type GNE cDNA can transduce murine muscle cells and human GNE myopathy muscle cells in culture and express the transgene in these cells. It was not clear that these cells could be successfully transduced, given their potential hyposialylated state and findings that some AAV viral vector types infect cells through sialylated receptors (Wu et al., 2006).

### EXAMPLE 2

### AAV/hGNE can be Used to Sucessfully Express GNE in Mice

The results of a pilot in vivo experiment, where mice were injected with AAV8/hGNE, either into muscle or intravenously and subsequent followed up for 35 days, indicated that human GNE mRNA is expressed either locally or systemically for the entire period. No adverse pathological effects or toxicity were detected.

These results prompted the design of a long-term experiment, 5-6 week old C57BL/6 mice were injected in the tail vein with either AAV8-hGNE-IRES-GFP (8.5×10¹¹ vg/mouse), AAV8-luciferase-IRES-GFP (8.5×10¹¹ vg/mouse), AAV8-luciferase-IRES-GFP at a higher dose (2.5×10¹² vg/mouse) or PBS (n=4).

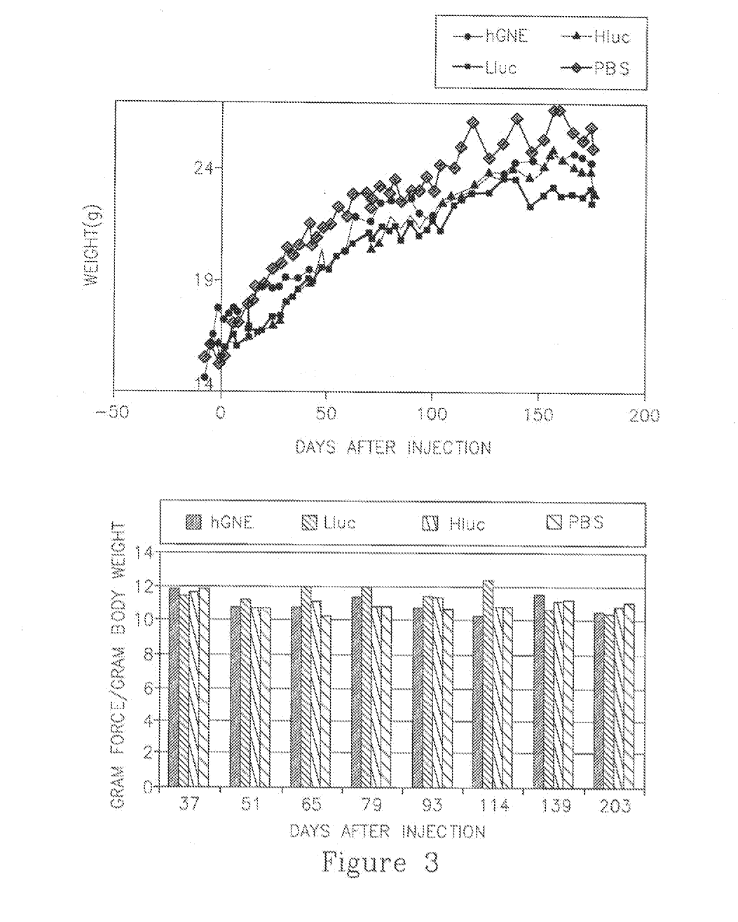

The mice were monitored over a 6-month period and their weight, behavior and grip force were examined at different time point. No statistically significant difference in these parameters was detected between the 4 groups of mice. (FIG. 3) during the entire period of follow up.

### Luciferase Activity

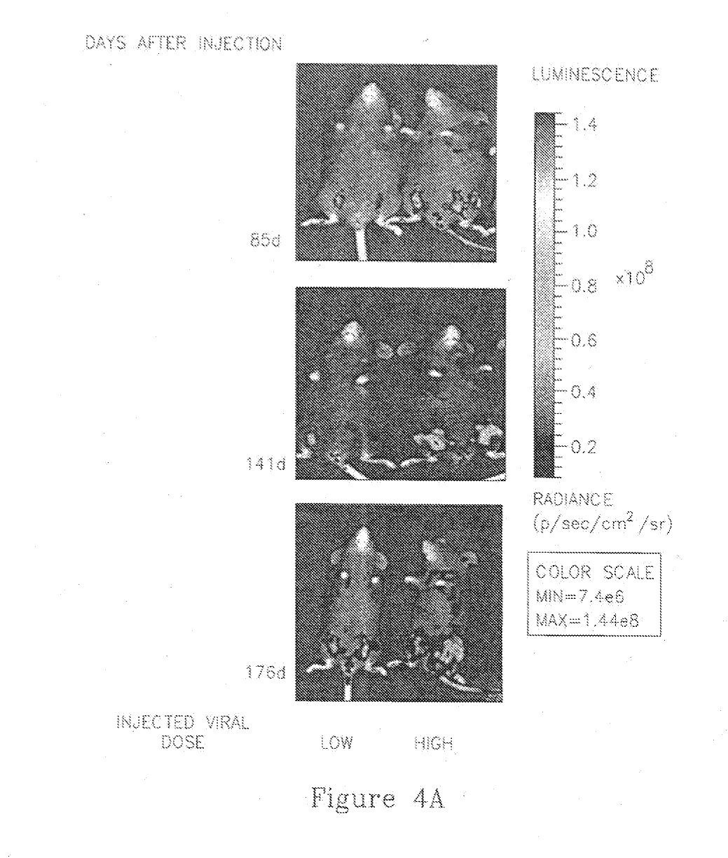

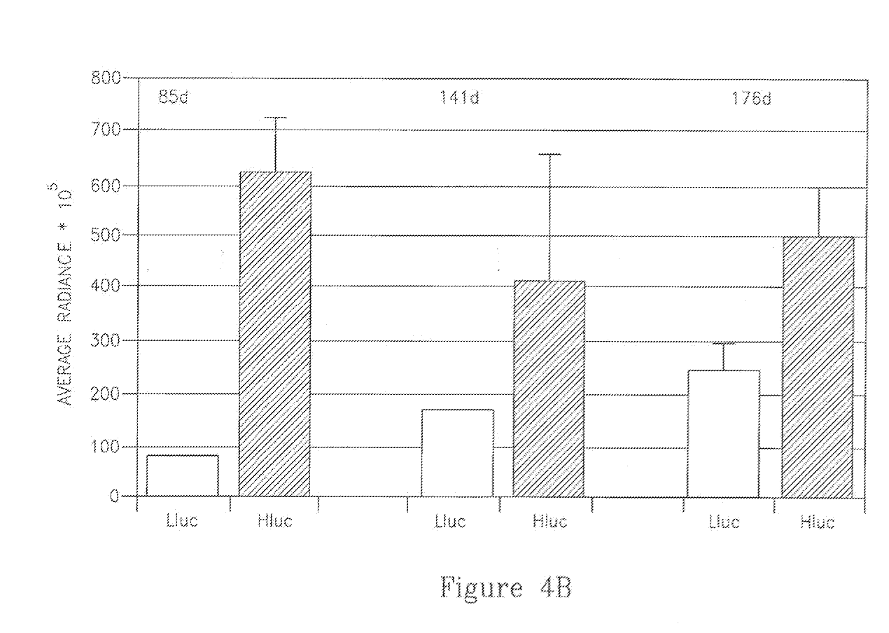

Luciferase activity was measured in mice injected with AAV8-luciferase-IRES-GFP at days 85, 141 and 176 after injection. Luciferase imaging revealed sustained luciferase activity during the entire period of observation (FIG. 4), and was stronger in mice injected with the higher viral dose (2.5×10¹² vg of AAV8-luciferase viral vector).

These findings demonstrate that AAV-mediated gene transfer is effective in vivo.

### EXAMPLE 3

### AAV/hGNE Vectors Enable Long-Term Expression of GNE in Muscle Tissues

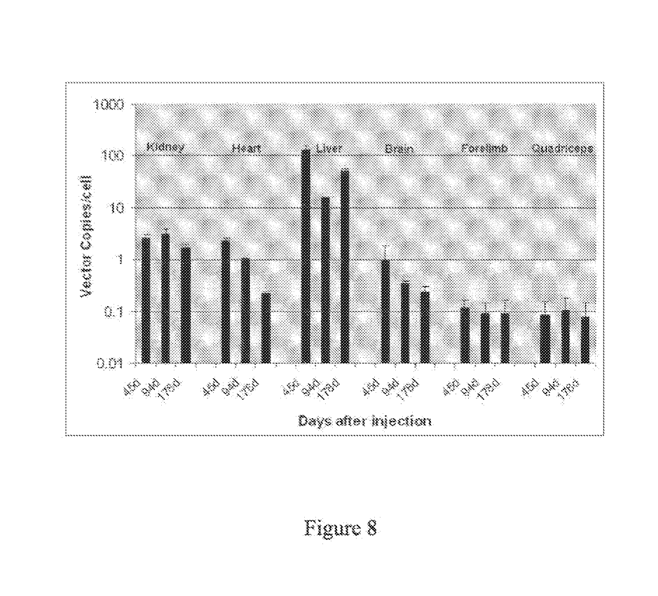

Mice in each group were sacrificed at 45, 94 and 178 days after injection, DNA from liver, kidney, heart, brain, forelimb and quadriceps of mice injected with AAV8/hGNE-IRES-GFP was analyzed for viral copy number (FIG. 8), and the tissues were analyzed by histology (H&E) for inflammation and tissue damage, and by real time PCR. The viral biodistribution was highest in liver (between 10-100 viral copies per cell), lower in kidney and heart (less than to 10 viral copies per cell), and lowest in brain (less than 1 viral copy per cell) and skeletal muscle (approximately 0.1 copy per cell in forelimb and quadriceps). These values were relatively stable, in particular in muscle tissue, with minimal time-related variation. Thus, the AAV8/hGNE viral copy number is stable in muscle tissue.

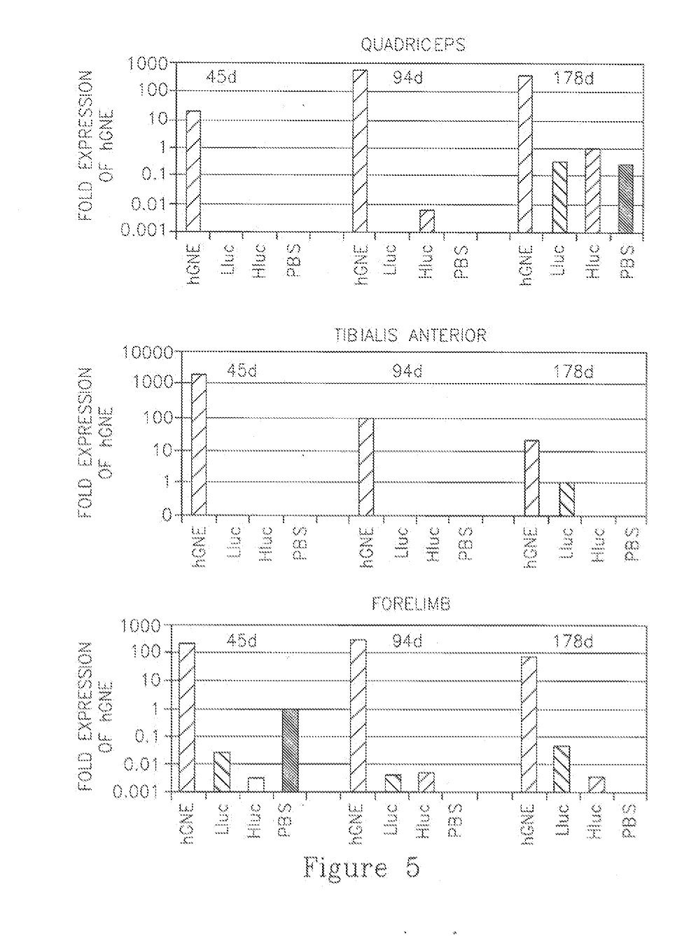

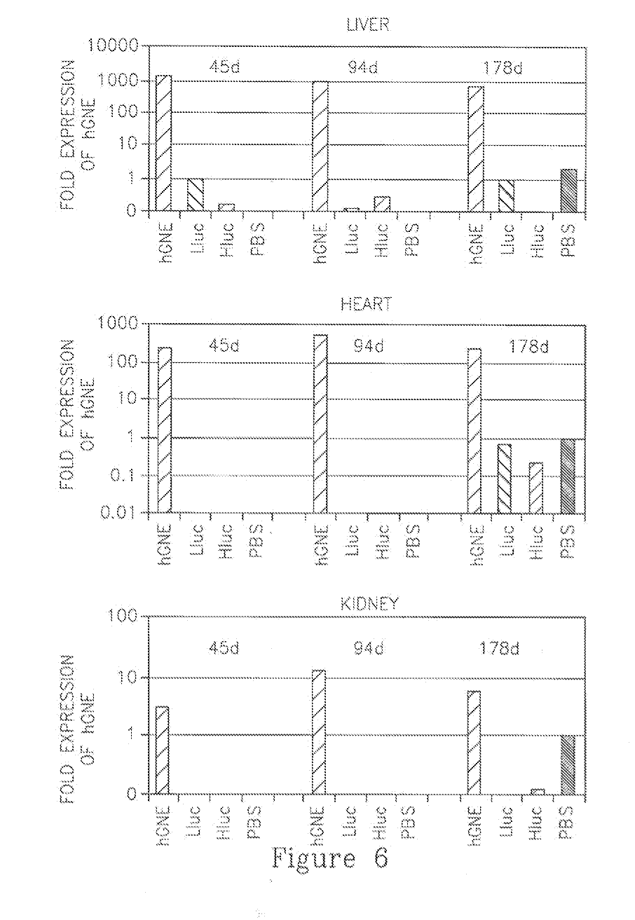

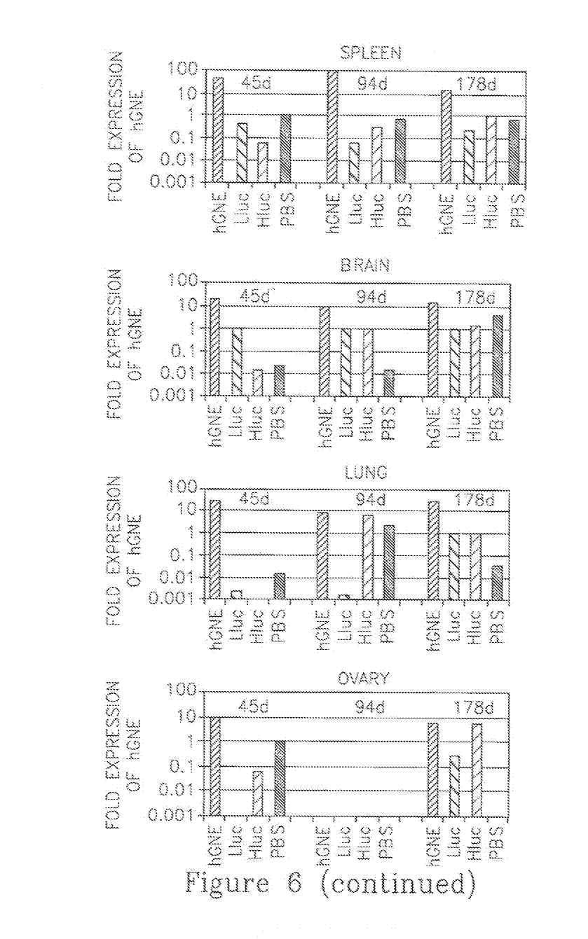

Quantitative real-time PCR analysis revealed that human GNE mRNA was still expressed 6 months after injection in skeletal muscles (FIG. 5) and in all other tissues examined (FIG. 6). In particular, skeletal muscles (quadriceps, tibialis anterior and forelimb) expressed hGNE from 10- to several hundred-fold greater than the value of mGNE measured in the same tissue. Liver and heart exhibited the same effect. In contrast, all other examined organs (kidney, spleen, brain, lung and ovary) expressed the injected gene to a much lower extent. Although a slight decrease in expression could be seen at the end point of the experiment (178 d post-injection), the expression was well-sustained during the entire experiment. As expected, there was no significant change in the expression of mouse GNE mRNA among the 4 experimental groups during this period.

Thus, AAV8-GNE was able to transduce mouse cells in vivo with sufficient efficiency to mediate in vivo long-term GNE expression. Moreover, although the viral vector was injected intravenously, GNE was expressed in muscle cells.

### EXAMPLE 4

### Histopathological Analysis Reveals No Pathological Changes in AAV/hGNE-Injected Mice

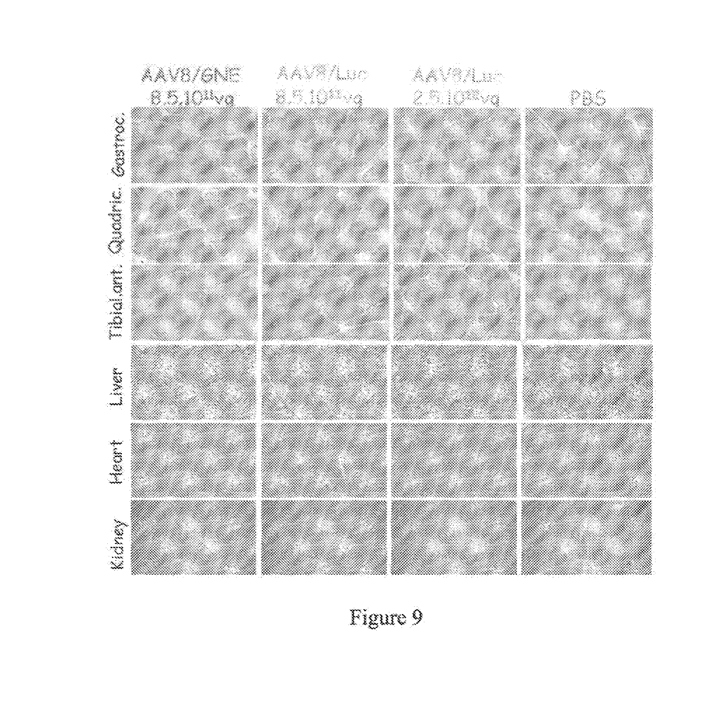

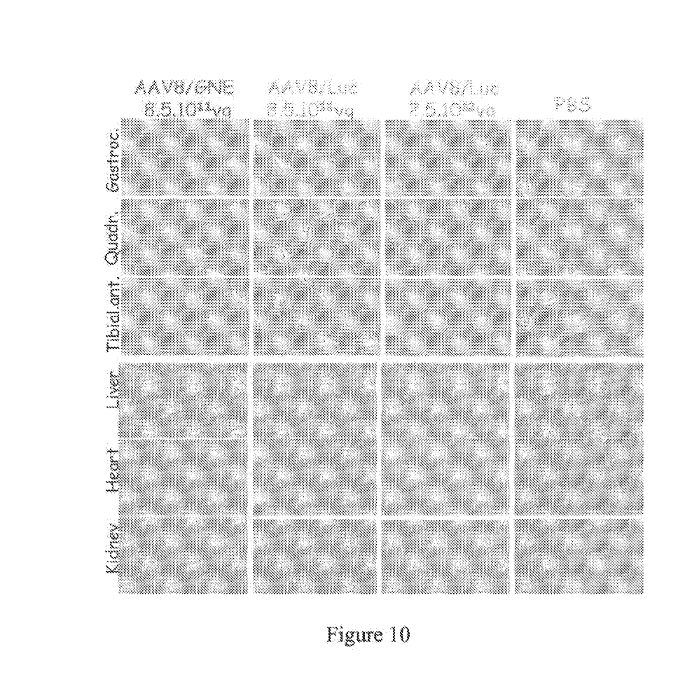

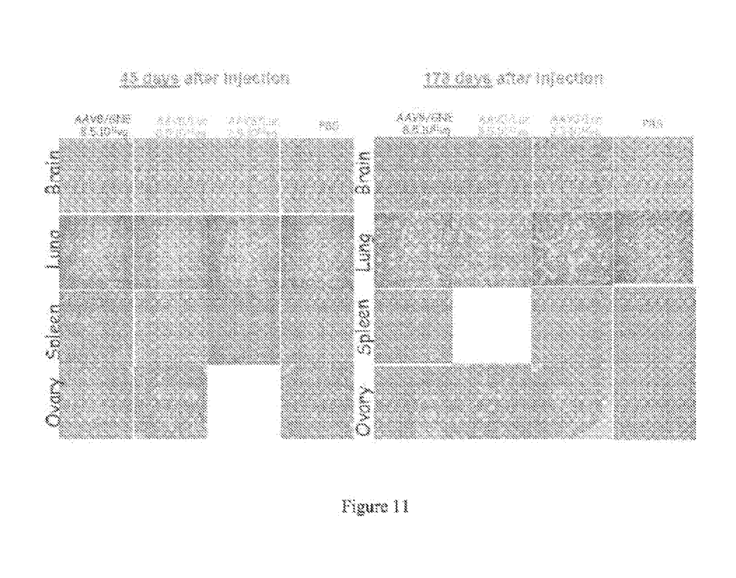

Histology (H&E) detected no pathological changes in any of the tissues analyzed, including liver, kidney, heart and the different muscles, at any of the 3 selected time points during the examination period. Additionally, no signs of inflammation were detected in the tissue sections (FIGS. 9-10). Other tissues were also examined such as lung, brain, spleen and ovary in females, with the same normal results (FIG. 11).

### EXAMPLE 5

### AAV/hGNE is Immunologically Well Tolerated

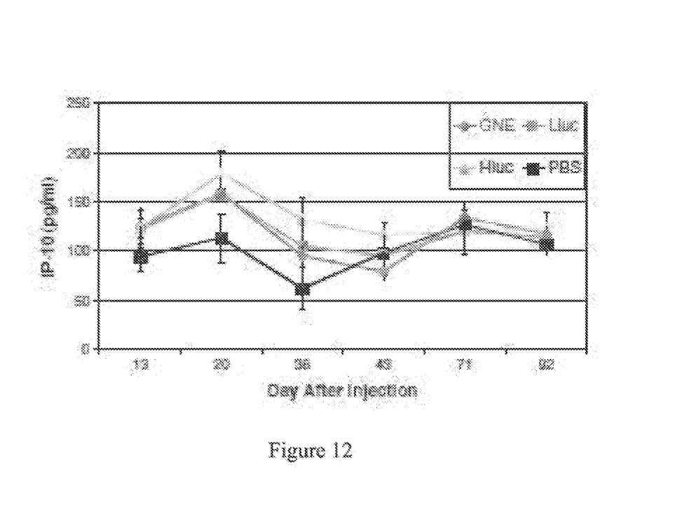

Serum was collected from all mice at different time points (from 13-92 days after injection) and assayed for expression of the inflammation marker IP-10 interferon gamma inducible protein) (Liu et al., 2011) by ELISA. As seen in FIG. 12, a mild increase in IP-10 could be detected around day 13, similar for all mice injected with viral vector compared to PBS-injected mice. This indicator of inflammatory response increased to a maximum on day 20 and decreased afterwards, remaining close to the baseline levels. A stronger response was observed in mice injected with a higher viral dose, 2.5×10¹² vg, of AAV2/8-luciferase viral vector. Thus, this transient response is apparently elicited by the viral capsids, independently of the cloned transgene.

Systemic injections of 8.5×10¹¹ vg/mouse and 2.5×10¹² vg/mouse were not toxic for the mice over the 6-month post-administration period. No adverse effects were observed in any organ analyzed; histologically and, as assessed by weight, motor force and behavior, all mice appeared completely healthy.

### CONCLUSIONS: EXAMPLES 1-5

Thus, AAV8-GNE was able to transduce mice with sufficient efficiency to mediate in vivo expression of GNE. Moreover, although the viral vector was injected intravenously, GNE was expressed in muscle cells. Additionally, the expression was sustained for at least 6 months. GNE was not directly assayed, due to the lack of a reliable and specific anti-GNE antibody; rather, mRNA GNE expression was demonstrated. It is highly likely that the GNE protein is also translated efficiently in these transduced mice.

A transient, mild increase in inflammatory markers was observed, with no abnormalities of any type observed. Thus, multi-systemic mRNA over-expression of wild-type human GNE was not deleterious in normal mice and is expected to be safe in GNE myopathy as well. These findings constitute an AAV-mediated therapy model and support the use of an AAV8-based vector for safe and efficient muscle therapy in GNE myopathy.

### EXAMPLE 6

### Testing of AAV/hGNE Vectors in an Animal Disease Model

A relevant animal model for GNE myopathy is a transgenic mouse model generated on a GNE^{-/-} background and over-expressing, the D176V GNE missense mutation occurring in the epimerase domain of the enxyme (the “DMRV/hIBM mouse model”; see Malicdan et al., 2007 and Malicdan et al., 2009). AAV8-based vectors that carry either human wt GNE or luciferase, as described in previous Examples, were injected intravenously into adult and symptomatic DMR/hIBM mice. Unaffected littermates were also injected as a control. At 10 weeks after injection, eGFP expression was seen in remarkable number of cells in the skeletal muscle, liver, kidney, heart, and spleen. Measurement of mRNA with specific human versus mouse probes revealed an increase in virus-derived human GNE expression. More importantly, DMRV/hIBM mice injected with AAV2/8-wt hGNE at 47 weeks of age showed a significant improvement in survival, motor performance, muscle size and contractile properties, as compared to those mice injected with AAV8-luciferase. These results show the efficacy AAV-mediated gene therapy for GNE myopathy.

### EXAMPLE 7

### AAV-Based Vectors Expressing hGNE From a Muscle-Specific Promoter

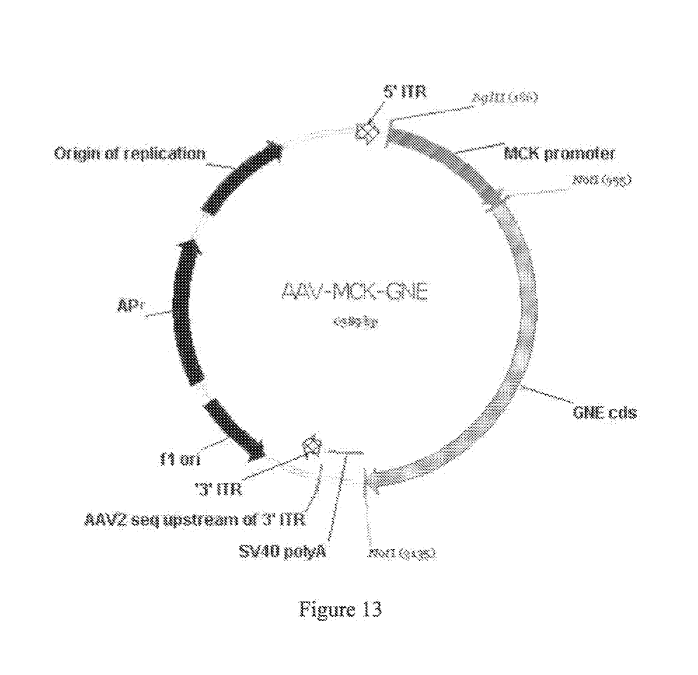

An AAV8 vector expressing hGNE with a muscle-specific promoter, AAV-MCK-hGNE (FIG. 13; SEQ ID NO: 29; the GNE cDNA spans nt 964-3133), was constructed as follows:

The MCK fragment was amplified from a plasmid provided by Dr Mendell at The Research Institute at Nationwide Children's Hospital, Columbus, Ohio, USA, and cloned into the backbone used for the previous vector by replacing the CMV-Luciferase-IRES-GFP segment. Subsequently, hGNE cDNA was cloned into it to generate the final vector.

Starting with the previous pCMV-GNE-IRES-eGFP plasmid, the CMV promoter was replaced by the MCK promoter, and the IRES sequences and GFP marker were excised.

The vector was transfected into HEK293 and C2C12 cells and found to express mRNA GNE in these cells. Subsequently, small scale virus was generated by triple transfection of HEK 293 cells by standard procedures (Matsushita et al., 1998). Virus was harvested after 72 hours by freeze/thaw cycles followed by centrifugation. The 3 plasmids used were the newly generated pMCK-hGNE plasmid, pRepCapAAV2/8 plasmid provided by Penn Vector Core at University of Pennsylvania, and the pHelper plasmid from Stratagene. Large-scale-purified pMCK-hGNE and pCMV-hGNE-IRES-GFP viral vectors used for mice intravenous injection were produced and titrated by viral genome (vg) determination.

### EXAMPLE 8

### Expression Studies of AAV-Based Vectors Expressing Muscle-Specific hGNE

Large scale production of the pMCK-GNE vector in an AAV8 capsid was performed at the Viral Vector Core at the Center for Gene Therapy, at The Research Institute at Nationwide Children's Hospital at Columbus, Ohio. 1×10¹² vg of this viral vector, in parallel to the above-described CMV vector, was injected into normal mice, and expression of human GNE mRNA was monitored in tibialis anterior (TA) muscle, liver, heart and kidney, 45 days after injection. Experiments were performed as described in previous Examples.

Quantification of hGNE expression was relative to the value detected in the corresponding tissue of PBS/control injected mice). All measurements were performed in duplicate and normalized relative to endogenous mouse GNE expression

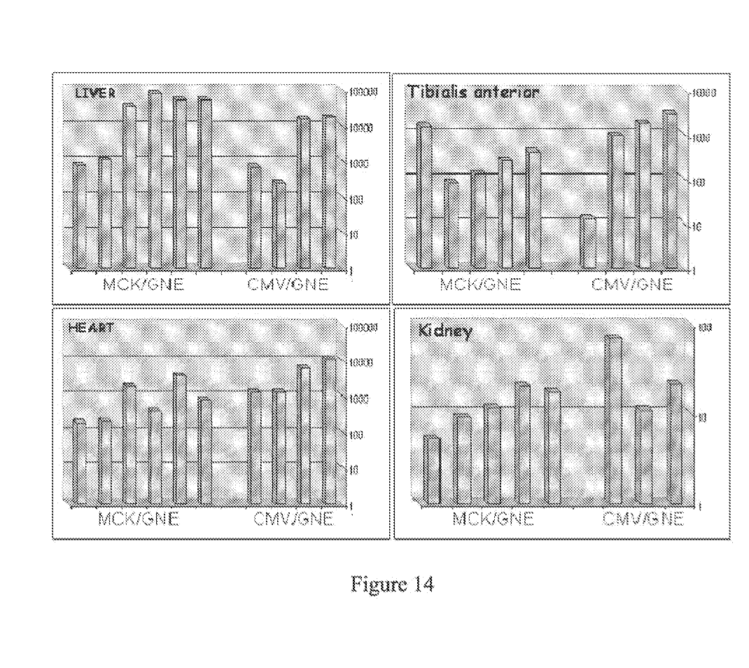

Both constructs were well expressed in the analysed tissues. The MCK promoter-based vector construct directed expression as well as the CMV promoter construct (FIG. 14). Vectors directing muscle-specific expression are expected to be superior to non-muscle-specific vectors such as CMV in treatment of myopathies.

### EXAMPLE 9

### Animal Model Testing of AAV-Based Vectors Expressing Muscle-Specific hGNE

The viral vectors are administered to GNE myopathy-model animals. In some experiments, administration of the vectors is performed at different time points of the animals' life span, for example before and after the expected onset of GNE myopathy symptoms, to ascertain whether the vector and prevent the appearance of GNE myopathy symptoms and/or can rescue animals symptoms. Animals are followed and compared to affected non-treated littermates for general behavior and clinical symptoms, appearance or disappearance of muscle weakness, and later sacrificed for analysis of human GNE expression in various tissues and for histological observation of the different tissues. In various experiments, muscle creatine kinase (CKM-promoter based vectors, or vectors using the promoters from a myosin light chain (MLC) promoter, for example MLC2, a myosin heavy chain (MHC) promoter, for example alpha- MHC, a desmin promoter, a cardiac troponin C promoter, a troponin I promoter, a myoD gene family promoter, an actin promoter, or the muscle-specific promoter residing within intron 1 of the ocular form of pitx3 are utilized.

### EXAMPLE 10

### Efficacy of AAV-Based Vectors Expressing Muscle-Specific hGNE in Treating Human Myopathies

The viral vectors are administered to humans afflicted with a GNE myopathy. In some experiments, administration of the vectors is performed at different points in the disease progression. Subjects are followed to determine tolerability of the therapy and are studied for clinical symptoms and disease progression in general, for example by measuring skeletal muscle strength in the limbs and/or other organs. In various experiments, different muscle-specific-promoter based vectors are utilized, for example as described hereinabove.

In some experiments, delivery of the viral vectors in humans is systemic, for example by intravenous injection. In other embodiments, viral vectors are delivered by locoregional injections to the limbs (either intravenous or intra-arterial), using a tourniquet for a short period of time to block the dissemination of the particles to the liver and favor their dissemination in the target limb muscles. This is expected to enhance the specificity conferred by the use of muscle-specific promoters.

It will be apparent that the precise details of the methods and compositions described herein may be varied or modified without departing from the spirit of the described invention. We claim all such modifications and variations that fall within the scope and spirit of the claims below, including all equivalents thereof.

In the claims, the word “comprise”, and variations thereof such as “comprises”, “comprising”, and the like indicate that the components listed are included, but not generally to the exclusion of other components.

### REFERENCES

Amsili, S, Zer, H, Hinderlich, S, Krause, S, Becker-Cohen, M, MacArthur, D G et al. (2008). UDP-N acetyl glucosamine 2-epimerase/N-acetylmannosamine kinase (GNE) binds to alpha-actinin 1: novel pathways in skeletal muscle? PLoS One 3: e2477.
Argov, Z and Yarom, R (1984). Rimmed vacuole myopathy sparing the quadriceps: A unique disorder in Iranian Jews. J Neurol Sci 64: 33-43.
Askanas, V and Engel, W K (1908). Newest approaches to diagnosis of sporadic inclusion body myositis and hereditary inclusion body myopathies, including molecular-pathologic similarities to Alzheimer disease. In: Inclusion Body Myositis and Myopathies, Cambridge, Cambridge University Press. 3-78.
Carter, B. J. (2005). Adeno-associated virus vectors in clinical trials. Hum Gene Ther 16, 541-550.
Coulon V et al, A muscle-specific promoter directs Pitx3 gene expression in skeletal muscle cells. J Biol Chem. 2007 Nov. 9; 282(45):33192-200.
Gadalla K K et al, Improved Survival and Reduced Phenotypic Severity Following AAV9MECP2 Gene Transfer to Neonatal and Juvenile Male Mecp2 Knockout Mice. Mol Ther. 2012.
Gao, G. P et al (2002). Novel adeno-associated viruses from rhesus monkeys as vectors for human gene therapy. Proc Natl Acad Sci USA 99, 11854-11859.
Hu, C, Busuttil, R W and Lipshutz G. S. (2010). Rh10 provides superior transgene expression in mice when compared with natural AAV serotypes for neonatal gene therapy. J Gene Med 12: 766-778.
Keppler O T et al. UDP-GlcNAc 2-epimerase: a regulator of cell surface sialylation, Science. 1999 May 21; 284(5418):1372-6.
Krause, S et al. (2007). GNE protein expression and subcellular distribution are unaltered in HIBM. Neurology 69: 655-659.
Little, S (1995). Clinical molecular genetics. Amplification Refractory Mutation System (ARMS) analysis of point mutations. In: Dracopoli N C, Haines J L, Korf B R (eds). Current Protocols in Human Genetics. Wiley & sons: New York., pp 9.8.1-9.8.2.
Liu, M, Guo, S, Hibbert, J M, Jain, V, Singh, N, Wilson, N O et al. (2011). CXCL10/IP-10 in infectious diseases pathogenesis and potential therapeutic implications. Cytokine Growth Factor Rev. 2011; 22:121-30.
Lochmuller, H, Johns, T and Shoubridge, E A (1999). Expression of the E6 and E7 genes of human papillomavirus (HPV16) extends the life span of human myoblasts, Exp Cell Res 248: 186-193.
Maguire, A. M., Simonelli, F., Pierce, E. A., Pugh, E. N., Jr., Mingozzi, F., Bennicelli, J., Banfi, S., Marshall, K. A., Testa, F., Surace, E. M., et al. (2008). Safety and efficacy of gene transfer for Leber's congenital amaurosis. N Engl J Med 358, 2240-2248.
Malicdan, M C, Noguchi, S, Nonaka, I, Hayashi, Y K and Nishino, I (2007). A Gne knockout mouse expressing human GNE DI76V mutation develops features similar to distal myopathy with rimmed vacuoles or hereditary inclusion body myopathy. Hum Mol Genet 16: 2669-2682.
Malicdan, M C, Noguchi, S, Hayashi, Y K, Nonaka, I and Nishino, I (2009). Prophylactic treatment with sialic acid metabolites precludes the development of the myopathic phenotype in the DMRV-hIBM mouse model. Nat Med 15: 690-5.
Markusic, D M, Herzog, R W, Aslanidi, G V, Hoffman, B E, Li, B, M et al. (2010). High-efficiency transduction and correction of murine hemophilia B using AAV2 vectors devoid of multiple surface-exposed tyrosines. Mol Thera 18: 2048-56.
Matsushita, T, Elliger, S, Elliger, C, Podsakoff, G, Villarreal, L, Kurtzman, G J et al. (1998). Adeno-associated virus vectors can be efficiently produced without helper virus. Gene Therapy 5: 938-945.
McCarty, D. M. (2008). Self-complementary AAV vectors; advances and applications. Mol Ther 16, 1648-1656.
McIntosh, J H, Cochrane, M, Cobbold, S, Waldmann, H, Nathwani, S A, Davidoff, A M et al. (2011). Successful attenuation of humoral immunity to viral capsid and transgenic protein following AAV-mediated gene transfer with a non-depleting CD4 antibody and cyclosporine Gene Therapy.
Mendell, J R, Rodino-Klapac, L R, Rosales, X Q., Coley, B D, Galloway, G, Lewis, S et al. (2010). Sustained Alpha-Sarcoglycan Gene Expression after Gene Transfer in Limb-Girdle Muscular Dystrophy, Type 2D). Ann Neurol 68: 629-638.
Nathwani A C et al, Long-term safety and efficacy following systemic administration of a self-complementary AAV vector encoding human FIX pseudotyped with serotype 5 and 8 capsid proteins. Mol Ther. 2011 May; 19(5):876-85.
Nemunaitis, G, Jay, C M, Maples, P B, Gahl, W A, Huizing, M, Yardeni, T et al. (2011). Hereditary Inclusion Body Myopathy: Single Patient Response to Intravenous Dosing of GNE Gene Lipoplex, Hum Gene Ther.
Nonaka, I, Sunohara, N, Ishiura, S and Satoyoshi (1981). Familial distal myopathy with rimmed vacuole and lamellar (myeloid) body formation. J Neurol Sci 51:141-153, 1981.
Park, K., Kim, W. J., Cho, Y. H., Lee, et al. (2008). Cancer gene therapy using adeno-associated virus vectors. Front Biosci 13, 2653-2659.
Wu, Z, Miller, E, Agbandje-McKenna, M and Samulski, R J (2006). Alpha2,3 and alpha2,6 N-linked sialic acids facilitate efficient binding and transduction by adeno-associated virus types 1 and 6. J Virol 80: 9093-103.

1. An adeno-associated virus (AAV)-based viral vector, comprising a nucleotide sequence encoding a UDP-N acetylglucosamine 2 epimerase/N-acetylmannosamine kinase (GNE) functionally linked to a muscle-specific promoter.

2. The AAV viral vector of claim 1, wherein said viral vector is selected from AAV serotypes 1, 2, 3, 4, 5, 6, 7, 8, 9, 10, and 11.

3. The AAV viral vector of claim 1, wherein said GNE is a human GNE.

4. The AAV viral vector of claim 1, wherein said GNE is a fully-functional GNE.

5. The AAV viral vector of claim 1, wherein said nucleotide sequence is a cDNA.

6. The AAV viral vector of claim 1, wherein said muscle-specific promoter is selected from the group consisting of a muscle creatine kinase (CKM)-promoter, a myosin light chain (MLC) promoter, a myosin heavy chain (MHC) promoter, a desmin promoter, a cardiac troponin C promoter, a troponin I promoter, a myoD gene family promoter, an actin promoter, and a promoter residing within intron 1 of the ocular form of pitx3.

7. The AAV viral vector of claim 1, wherein said viral vector exhibits reduced immunogenicity.

8. A host cell comprising the AAV viral vector of claim 1.

9. A pharmaceutical composition comprising the AAV viral vector of claim 1.

10. The pharmaceutical composition of claim 9 for treating a myopathy associated with deficient GNE function.

11. The pharmaceutical composition of claim 10, wherein said myopathy is selected from the group consisting of hereditary inclusion body myopathy (HIBM), quadriceps sparing myopathy, distal myopathy with rimmed vacuoles (DMRV) and Nonaka's disease.

12. The pharmaceutical composition of claim 11, wherein said myopathy is an established myopathy.

13. The pharmaceutical composition of claim 10, wherein a single administration of said pharmaceutical composition confers lasting expression of said GNE in a subject having said myopathy.

14. The pharmaceutical composition of claim 9, wherein said pharmaceutical composition is indicated for systemic administration; for locoregional administration in a limb, in conjunction with restriction of the venous circulation of the treated limb; or both.

15. (canceled)

16. The pharmaceutical composition of claim 9, wherein said pharmaceutical composition is indicated for administration together with immunosuppressive therapy.

17. A method of treating a myopathy associated with deficient GNE function in a subject in need thereof, comprising the step of administering a pharmaceutical composition comprising the AAV viral vector of claim 1, thereby treating a myopathy associated with deficient GNE function.

18. The method of claim 17, wherein said myopathy is selected from the group consisting of hereditary inclusion body myopathy (HIBM), quadriceps sparing myopathy, distal myopathy with rimmed vacuoles (DMRV) and Nonaka's disease, or wherein said myopathy is an established myopathy.

19. (canceled)

20. The method of claim 17, wherein a single administration of said pharmaceutical composition confers lasting expression of said GNE in a subject having said myopathy.

21. The method of claim 17, wherein said pharmaceutical composition is injected systemically; is administered locoregionally in a limb, in conjunction with restriction of the venous circulation of the treated limb; or both.

22. (canceled)

23. The method of claim 17, wherein said pharmaceutical composition is administered together with immunosuppressive therapy.
